SEQUENCE LISTING (9 sequences)

SEQ ID NO: 1 - DNA, 4100 bp, Homo sapiens
ggaaaccgagcgggcccatttttcatgggtttgcggacccaccagcgaaggcgggaggtgtcgcagggacatcttctggctgtttccgtcgcctgcgtggcccttgcaccccggtcttccattagcggcgcagacgtttgggcctaagcgctgggcgaggcgaggccctgcccctccccgccaacggccattctctggacctgtctttcttccgggaggcggtgacagctgctgagacgtgttgcagccagagtctctccgctttaatgcgctcccattagtgccgtcccccactggaaaaccgtggcttctgtattatttgccatctttgttgtgtaggagcagggagggcttcctcccggggtcctaggcggcggtgcagtccgtcgtagaagaattagagtagaagttgtcggggtccgctcttaggacgcagccgcctcatgggggtccaggggctctggaagctgctggagtgctccgggcggcaggtcagccccgaagcgctggaagggaagatcctggctgttgatattagcatttggttaaaccaagcacttaaaggagtccgggatcgccatgggaactcaatagaaaatcctcatcttctcactttgtttcatcggctctgcaaactcttattttttcgaattcgtcctatttttgtgtttgatggggatgctccactattgaagaaacagactttggtgaagagaaggcagagaaaggacttagcgtccagtgactccaggaaaacgacagagaagcttctgaaaacatttttgaaaagacaagccatcaaaactgccttcagaagcaaaagagatgaagcactacccagtcttacccaagttcgaagagaaaacgacctctatgttttgcctcctttacaagaggaagaaaaacacagttcagaagaggaagatgaaaaagaatggcaagaaagaatgaatcaaaaacaagcattacaggaagagttctttcataatcctcaagcgatagatattgagtctgaggacttcagcagcctgccccctgaagtaaagcatgaaatcttgactgatatgaaagagttcaccaagcgcagaagaacattatttgaagcaatgccagaggagtctgatgacttttcacagtaccaactcaaaggcttgcttaaaaagaactatctgaaccagcatatagaacatgtccaaaaggaaatgaatcagcaacattcaggacacatccgaaggcagtatgaagatgaagggggctttctgaaggaggtagagtcaaggagagtggtctctgaagacacttcacattacatcttgataaaaggtattcaagctaagacagttgcagaagtggattcagagtctcttccttcttccagcaaaatgcacggcatgtcttttgacgtgaagtcatctccatgtgaaaaactgaagacagagaaagagcctgatgctacccctccttctccaagaactttactagctatgcaagctgccctgctgggaagtagctcagaagaggagctggagagtgaaaatcgaaggcaggcccgtgggaggaacgcacctgctgctgtagacgaaggctccatatcaccccggactctttcagccattaagagagctcttgacgatgacgaagatgtaaaagtgtgtgctggggatgatgtgcagacgggagggccaggagcagaagaaatgcgtataaacagctccaccgagaacagtgatgaaggacttaaagtgagagatggaaaaggaataccgtttactgcaacacttgcgtcatctagtgtgaactctgcagaggagcacgtagccagcactaatgaggggagagagcccacagactcagttccaaaagaacaaatgtcacttgttcacgtggggactgaagcctttccgataagtgatgagtctatgattaaggacagaaaagatcggctgcctctggagagtgcagtggttagacatagtgacgcacctgggctcccgaatggaagggaactgacaccggcatctccaacttgtacaaattctgtgtcaaagaatgaaacacatgctgaagtgcttgagcagcagaacgaactttgcccatatgagagtaaattcgattcttctcttctttcaagtgatgatgaaacaaaatgtaaaccgaattctgcttctgaagtcattggccctgtcagtttgcaagaaacaagtagcatagtaagtgtcccttcagaggcagtagataatgtggaaaatgtggtgtcatttaatgctaaagagcatgagaattttctggaaaccatccaagaacagcagaccactgaatctgcaggccaggatttaatttccattccaaaggccgtggaaccaatggaaattgactcggaagaaagtgaatctgatggaagtttcattgaagtgcaaagtgtgattagtgatgaggaacttcaagcagaattccctgaaacttccaaacctccctcagaacaaggcgaagaggaactggtaggaactagggagggagaagcccctgctgagtccgagagcctcctgagggacaactctgagagggacgacgtggatggtgagccacaggaagctgagaaagatgcggaagattcgctccatgaatggcaagatattaatttggaggagttggaaactctggagagcaacctcttagcacagcagaattcactgaaagctcaaaaacagcagcaagaacggatcgctgctactgtcaccggacagatgttcctggaaagccaggaactcctgcgcctgttcggcattccctacatccaggctcccatggaagcagaggcgcagtgcgccatcctggacctgactgatcagacttccggaaccatcactgatgacagtgatatctggctgtttggagcgcggcatgtctatagaaacttttttaataaaaacaagtttgtagaatattatcaatatgtggactttcacaatcaattgggattggaccggaataagttaataaatttggcttatttgcttggaagtgattataccgaaggaataccaactgtgggttgtgtaaccgccatggaaattctcaatgaattccctgggcatggcctggaacctctcctaaaattctcagaatggtggcatgaagctcaaaaaaatccaaagataagacctaatcctcatgacaccaaagtgaaaaaaaaattacggacattgcaactcacccctggctttcctaacccagctgttgccgaggcctacctcaaacccgtggtggatgactcgaagggatcctttctgtgggggaaacctgatctcgacaaaattagagaattttgtcagcggtatttcggctggaacagaacgaagacagatgaatctctgtttcctgtattaaagcaactcgatgcccagcagacacagctccgaattgattccttctttagattagcacaacaggagaaagaagatgctaaacgtattaagagccagagactaaacagagctgtgacatgtatgctaaggaaagagaaagaagcagcagccagcgaaatagaagcagtttctgttgccatggagaaagaatttgagctacttgataaggcaaaacgaaaaacccagaagagaggcataacaaataccttagaagagtcatcaagcctgaaaagaaagaggctttcagattctaaacgaaagaatacatgcggtggatttttgggggagacctgcctctcagaatcatctgatggatcttcaagtgaagatgctgaaagttcatctttaatgaatgtacaaaggagaacagctgcgaaagagccaaaaaccagtgcttcagattcgcagaactcagtgaaggaagctcccgtgaagaatggaggtgcgaccaccagcagctctagtgatagtgatgacgatggagggaaagagaagatggtcctcgtgaccgccagatctgtgtttgggaagaaaagaaggaaactaagacgtgcgaggggaagaaaaaggaaaacctaattaaaaaatatgtatcctctataattagttatgacagccatttgtaatgaatttgtcgcaaagacgtaataaaattaactggtggcacggtcaaaa

SEQ ID NO: 2 - DNA, 18 bp, Artificial Sequence
  Description of Artificial Sequence Synthetic forward primer for amplifying beta-actin gene
tgagcgcggctacagctt

SEQ ID NO: 3 - DNA, 22 bp, Artificial Sequence
  Description of Artificial Sequence Synthetic reverse primer for amplifying beta-actin gene
tccttaatgtcacgcacgattt

SEQ ID NO: 4 - DNA, 18 bp, Artificial Sequence
  Description of Artificial Sequence Synthetic probe for detecting beta-actin gene
accaccacggccgagcgg

SEQ ID NO: 5 - DNA, 20 bp, Artificial Sequence
  Description of Artificial Sequence Synthetic forward primer for amplifying XPG gene
gaagcgctggaagggaagat

SEQ ID NO: 6 - DNA, 26 bp, Artificial Sequence
  Description of Artificial Sequence Synthetic reverse primer for amplifying XPG gene
gactcctttaagtgcttggtttaacc

SEQ ID NO: 7 - DNA, 21 bp, Artificial Sequence
  Description of Artificial Sequence Synthetic probe for detecting XPG gene
ctggctgttgatattagcatt

SEQ ID NO: 8 - DNA, 20 bp, Artificial Sequence
  Description of Artificial Sequence Synthetic forward primer used for genotyping of Asp1104His SNP at XPG gene
tggatttttgggggagacct

SEQ ID NO: 9 - DNA, 21 bp, Artificial Sequence
  Description of Artificial Sequence Synthetic reverse primer for genotyping of Asp1104His SNP at XPG gene
cgggagcttccttcactgagt

(19) United States
(12) Patent Application Publication
(10) Pub. No.: US 2010/0267732 A1
(43) Pub. Date: Oct. 21, 2010

(54) Prognostic Molecular Markers for ET-743 Treatment

(75) Inventors: Rafael Rosell Costa, Badalona (ES); Miguel Tarón Roca, Badalona (ES); Nerea Martínez Magunacelaya, Madrid (ES); Ana Díez Rodríguez, Madrid (ES); José Mª Jimeno Donaque, Madrid (ES); Juan Carlos Tercero López, Madrid (ES)

Correspondence Address: KING & SPALDING, 1185 AVENUE OF THE AMERICAS, NEW YORK, NY, 10036-4003, US

(73) Assignee: PHARMA MAR, S.A., Madrid (ES)

(21) Appl. No.: 12/738,722

(22) PCT Filed: Oct. 20, 2008
(86) PCT No.: PCT/EP08/64156

§ 371 Date: Apr. 19, 2010

(30) Foreign Application Priority Data
Oct. 19, 2007 (EP) 07380282.9

(51) Int. Cl. A61K 31/519; C12Q 1/68; A61P 35/00

(52) U.S. Cl. 514/250; 435/6

(57) ABSTRACT

The present invention relates to the use of ecteinascidin 743 in human patients having certain molecular markers profile which has been associated with the outcome of ET-743 chemotherapy. In particular, the invention relates to the use of ecteinascidin 743 in patients having high expression levels of XPG mRNA or protein and/or having a “wild type” genotype for Asp1104His SNP of XPG gene.

### FIELD OF THE INVENTION

The present invention relates to the use of ecteinascidin 743 (ET-743), and more especially to the use of ET-743 in human patients having tumours with certain molecular markers profile, in particular having high XPG mRNA or protein expression levels and/or having a C nucleotide in at least one of the alleles at the SNP locus is for Asp1104His SNP of XPG gene. The invention also relates to methods for providing personalised ET-743 chemotherapy to cancer patients based on said tumour molecular markers.

### BACKGROUND OF THE INVENTION

Cancer develops when cells in a part of the body begin to grow out of control. Although there are many kinds of cancer, they all start because of out-of-control growth of abnormal cells. Cancer cells can invade nearby tissues and can spread through the bloodstream and lymphatic system to other parts of the body. There are several main types of cancer. Carcinoma is cancer that begins in the skin or in tissues that line or cover internal organs. Epithelial cells, which cover internal and external surfaces of the body, including organs and lining of vessels, may give rise to a carcinoma. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system.

In addition, cancer is invasive and tends to metastasise to new sites. It spreads directly into surrounding tissues and also may be disseminated through the lymphatic and circulatory systems.

Many treatments are available for cancer, including surgery and radiation for localised disease, and chemotherapy. However, the efficacy of available treatments for many cancer types is limited, and new, improved forms of treatment showing clinical benefit are needed. This is especially true for those patients presenting with advanced and/or metastatic disease and for patients relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment or ultimately die from progressive metastatic disease. Thus, the ongoing commitment to the design and discovery of new anticancer agents is critically important.

Chemotherapy, in its classic form, has been focused primarily on killing rapidly proliferating cancer cells by targeting general cellular metabolic processes, including DNA, RNA, and protein biosynthesis. Chemotherapy drugs are divided into several groups based on how they affect specific chemical substances within cancer cells, which cellular activities or processes the drug interferes with, and which specific phases of the cell cycle the drug affects. The most commonly used types of chemotherapy drugs include: DNA-alkylating drugs (such as cyclophosphamide, ifosfamide, cisplatin, carboplatin, dacarbazine), antimetabolites (5-fluorouracil, capecitabine, 6-mercaptopurine, methotrexate, gemcitabine, cytarabine, fludarabine), mitotic inhibitors (such as paclitaxel, docetaxel, vinblastine, vincristine), anthracyclines (such as daunorubicin, doxorubicin, epirubicin, idarubicin, mitoxantrone), topoisomerase I and II inhibitors (such as topotecan, irinotecan, etoposide, teniposide), and hormone therapy (such as tamoxifen, flutamide).

The ideal antitumor drug would kill cancer cells selectively, with a wide index relative to its toxicity towards non-cancer cells and it would also retain its efficacy against cancer cells, even after prolonged exposure to the drug. Unfortunately, none of the current chemotherapies with these agents posses an ideal profile. Most posses very narrow therapeutic indexes and, in addition, cancerous cells exposed to slightly sublethal concentrations of a chemotherapeutic agent may develop resistance to such an agent, and quite often cross-resistance to several other antitumor agents.

The ecteinascidins (herein abbreviated ETs) are exceedingly potent antitumor agents isolated from the marine tunicate Ecteinascidia turbinata. Several ecteinascidins have been reported previously in the patent and scientific literature. See, for example U.S. Pat. No. 5,089,273, which describes novel compounds of matter extracted from the tropical marine invertebrate Ecteinascidia turbinata, and designated therein as ecteinascidins 729, 743, 745, 759A, 759B and 770. These compounds are useful as antibacterial and/or antitumor agents in mammals. U.S. Pat. No. 5,478,932 describes other novel ecteinascidins isolated from the Caribbean tunicate Ecteinascidia turbinata, which provide in vivo antitumor activity against P388 lymphoma, B16 melanoma, M5076 ovarian sarcoma, Lewis lung carcinoma, and the LX-1 human lung and MX-1 human mammary carcinoma xenografts.

One of the ETs, ecteinascidin 743 (ET-743), is a tetrahydroisoquinoline alkaloid with considerable in vitro and in vivo antitumor activity in murine and human tumors, and potent antineoplastic activity against a variety of human tumor xenografts grown in athymic mice, including melanoma, ovarian and breast carcinoma.

ET-743 is a natural compound with the following structure:

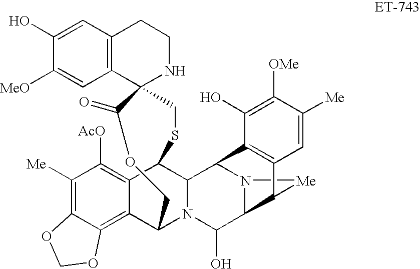

ET-743 is also known with the generic name trabectedin and the trademark Yondelis®, and it is currently approved in Europe for the treatment of soft tissue sarcoma. The clinical development of trabectedin continues in phase II/III clinical trials in breast, ovarian and prostate cancer. A clinical development program of ET-743 in cancer patients was started with phase I studies investigating 1-hour, 3-hour, 24-hour, and 72-hour intravenous infusion schedules and a 1 hour daily×5 (d×5) schedule. Promising responses were observed in patients with sarcoma, breast and ovarian carcinoma. Therefore this new drug is currently under intense investigation in several phase II/III clinical trials in cancer patients with a variety of neoplastic diseases. Further information regarding the dosage, schedules, and administration of ET-743 for the treatment of cancer in the human body, either given alone or in combination is provided in WO 00/69441, WO 02/36135, WO 03/39571, WO 2004/105761, WO 2005/039584, WO 2005/049031, WO 2005/049030, WO 2005/049029, WO 2006/046080, WO 2006/005602, and PCT/US07/98727, which are incorporated by reference herein in their entirety.

A review of ET-743, its chemistry, mechanism of action and preclinical and clinical development can be found in Kesteren, Ch. Van et al., Anti-Cancer Drugs, 2003, 14 (7), 487-502: “ET-743 (trabectedin, ET-743): the development of an anticancer agent of marine origin”, and references therein.

During the past 30 years medical oncologists have focused to optimise the outcome of cancer patients and it is just now that the new technologies available are allowing to investigate polymorphisms, gene expression levels and gene mutations aimed to predict the impact of a given therapy in different groups of cancer patients to tailor chemotherapy. Representative examples include the relationship between the Thymidylate Synthase (TS) mRNA expression and the response and the survival with antifolates, beta tubulin III mRNA levels and response to tubulin interacting agents, PTEN gene methylation and resistance to CPT-11 and, STAT3 over expression and resistance to Epidermal Growth Factor (EGF) interacting agents.

A molecular observation of potential clinical impact relates to the paradoxical relation between the efficiency of the Nucleotide Excision Repair (NER) pathway and the cytotoxicity of ET-743. In fact, tumour cells that are efficient in this DNA repair pathway appear to be more sensitive to ET-743. This evidence is in contrast with the pattern noted with platin based therapeutic regimens which are highly dependent on the lack of activity of this repair pathway (ie. an increase in ERCC1 expression has been associated to clinical resistance to platinum-based anti-cancer therapy).

There are evidences on the key role of NER pathways on the cytotoxicity of ET-743 in cell lines. ET-743 binds to G residues in the minor groove of DNA forming adducts that distort the DNA helix structure and they are recognised by NER mechanisms (Pourquier, P. et al., 2001, Proceedings of the American Association for Cancer Research Annual Meeting, Vol. 42, pp. 556. 92nd Annual Meeting of the American Association for Cancer Research. New Orleans, La., USA. Mar. 24-28, 2001. ISSN: 0197-016X). Takebayasi et al. (Nature Medicine, 2001, 7(8), 961-966) have proposed that the presence of these DNA adducts in transcribed genes, blocks the Transcription Coupled NER (TC-NER) system by stalling the cleavage intermediates and producing lethal Single Strand Breaks (SSBs). It is known from Grazziotin et al (Proc. Natl. Acad. Sic. USA, 104:13062-13067) that the DNA adducts formed by exposure to ET-743 are transformed into double strand DNA breaks.

The fact that NER mediates ET-743's cytotoxicity has also been found in the yeast Saccharomyces cerevisae by Grazziotin et al. (Biochemical Pharmacology, 2005, 70, 59-69) and in the yeast Schizosaccharomyces pombe by Herrero et al. (Cancer Res. 2006, 66(16), 8155-8162).

In addition, Bueren et al. (Proceedings AACR Annual Meeting 2007, Abstract no. 1965) have been shown that ET-743 induces double-strand breaks in the DNA in early S phase that are detected and repaired by the Homologous Recombination Repair (HRR) pathway. In addition, Erba et al (Eur. J. Cancer, 2001, 37(1), 97-105) and Bueren et al (Proceedings AACR Annual Meeting 2007, Abstract no. 1965) have shown that inactivation/mutations of genes related to the Double Strand Break detection such as DNA-PK, ATM and ATR and of genes related to Homologous Recombination Repair pathway, such as Fanconi Anemia genes, BRCA1, BRCA2 and RAD51 make cells more sensitive to trabectedin. Such unique finding is the opposite to the pattern with conventional DNA interacting agents, like in the case of microtubule poisons such as taxanes and vinorelbine.

Finally, pharmacogenomic studies prior have demonstrated that increased expression of the NER genes ERCC1 and XPD in the tumor tissue does not impact the outcome of patients treated with ET-743. However, the low expression of BRCA1 in the tumor tissue is correlated with a better outcome in cancer patents treated with ET-743. Further information can be found in WO 2006/005602, which is incorporated by reference herein in its entirety.

Three rare, autosomal recessive inherited human disorders are associated with impaired NER activity: xeroderma pigmentosum (XP), Cockayne Syndrome (CS), and trichothiodystrophy (Bootsma et al. The Genetic Basis of Human Cancer. McGraw-Hill, 1998, 245-274). XP patients exhibit extreme sensitivity to sunlight, resulting in a high incidence of skin cancers (Kraemer et al. Arch. Dermatol. 123, 241-250, and Arch. Dermatol. 130, 1018-1021). About 20% of XP patients also develop neurologic abnormalities in addition to their skin problems. These clinical findings are associated with cellular defects, including hypersensitivity to killing and mutagenic effects of UV, and inability of XP cells to repair UV-induced DNA damage (van Steeg et al. Mol. Med. Today, 1999, 5, 86-94).

Seven different NER genes, which correct seven distinct genetic XP complementation groups (XPA-XPG), have been identified (Bootsma et al. The Genetic Basis of Human Cancer. McGraw-Hill, 1998, 245-274). The human gene responsible for XP group G was identified as ERCC5 (Mudgett et al. Genomics, 1990, 8, 623-633; O'Donovan et al. Nature, 1993, 363, 185-188; and Nouspikel et al. Hum. Mol. Genet. 1994, 3, 963-967). The XPG gene codes for a structure-specific endonuclease that cleaves damaged DNA -5 nt 3′ to the site of the lesion and is also required non-enzymatically for subsequent 5′ incision by the XPF/ERCC1 heterodimer during the NER process (Aboussekhra et al. Cell, 1995, 80, 859-868; Mu et al. J. Biol. Chem. 1996, 271, 8285-8294; and Wakasugi et al. J. Biol. Chem. 1997, 272, 16030-16034). There is also evidence suggesting that XPG is also involved in transcription-coupled repair of oxidative DNA lesions (Le Page et al. Cell, 101, 159-171).

Takebayashi et al. (Cancer Lett., 2001, 174:115-125) have observed an increase in heterozygosity loss and microsatellite instability in a substantial percentage of samples of ovarian, lung and colon carcinoma. Le Moirvan et al, (Int. J. Cancer, 2006, 119:1732-1735) have described the presence of polymorphisms in the XPG gene in sarcoma patients. It is also known from Takebayashi et al. (Proceedings of the American Association for Cancer Research Annual Meeting, March, 2001, Vol. 42, pp. 813. 92nd Annual Meeting of the American Association for Cancer Research. New Orleans, La., USA. March 24-28, 2001) that cells deficient in the NER system are resistant to treatment with ET-743 (Zewail-Foote, M. et al., 2001, Chemistry and Biology, 8:1033-1049 and Damia, G. et al., 2001, Symposium AACR NCI EORTC) and that the antiproliferative effects of ET-743 require a functional XPG gene.

Since cancer is a leading cause of death in animals and humans, several efforts have been and are still being undertaken in order to obtain an antitumor therapy active and safe to be administered to patients suffering from a cancer. Accordingly, there is a need for providing additional antitumor therapies that are useful in the treatment of cancer.

### SUMMARY OF THE INVENTION

In a first aspect, the invention relates to a method of predicting the clinical response of a cancer patient to ET-743 chemotherapy comprising
a) determining the expression level of XPG mRNA in a biological sample of the patient before the ET-743 chemotherapy; and
b) comparing the amount of expression of XPG mRNA in the biological sample with the median value of the expression of XPG mRNA measured in a collection of biological samples
   wherein an expression level of XPG mRNA equal to or higher than the median value of expression levels of XPG mRNA is indicative that the patient will show a positive response after treatment with ET-743.

In another aspect, the invention relates to a method of predicting the clinical response of a cancer patient to ET-743 chemotherapy comprising
a) determining the expression level of XPG protein in a biological sample of the patient before the ET-743 chemotherapy; and
b) recording the results of the determination of the expression levels of XPG protein as negative expression (0), low expression (1+), moderate expression (2+), or high expression (3+)
   wherein moderate or high expression levels of XPG protein is indicative that the patient will show a positive response after treatment with ET-743.

In another aspect, the invention relates to a method of predicting the clinical response of a cancer patient to ET-743 chemotherapy comprising the step of determining the genotype of the Asp1104His SNP at locus rs17655 of the XPG gene in a biological sample of said patient before the ET-743 chemotherapy wherein the presence of a C nucleotide in at least one of the alleles at the SNP locus is indicative that the patient will show a positive response to the treatment with ET-743.

In yet another aspect, the invention relates to an in vitro method for designing an individual chemotherapy for a human patient suffering from cancer, comprising:
a) determining the expression level of XPG mRNA in a biological sample from said patient;
b) comparing the expression level of XPG mRNA obtained in a) with the median value of the expression level of XPG mRNA measured in a collection of tumor tissue in biopsy samples from human cancer patients; and
c) selecting a chemotherapy treatment based on ET-743 when said XPG mRNA expression level is equal to or above the median value of expression levels of XPG mRNA.

In yet another aspect, the invention relates to an in vitro method for designing an individual chemotherapy for a human patient suffering from cancer, comprising:
a) determining the expression level of the XPG protein in a biological sample from said patient;
b) recording the results of the determination under (a) as negative expression (0), low expression (1+), moderate expression (2+), or high expression (3+); and
c) selecting a chemotherapy treatment based on ET-743 when said XPG protein expression level has a value of (2+) or (3+).

In another aspect, the invention relates to an in vitro method for designing an individual chemotherapy for a human patient suffering from cancer, comprising:
a) determining the genotype of the Asp1104His SNP at locus rs17655 of the XPG gene in a biological sample from said patient; and
b) selecting a chemotherapy treatment based on ET-743 when a C nucleotide is present in at least one of the alleles at the SNP locus.

In another aspect, the invention relates to a screening method for selecting a human patient suffering from cancer for a treatment with ET-743, comprising the steps:
a) determining the expression level of XPG mRNA in a biological sample of the patient;
b) comparing the expression level of XPG mRNA obtained in a) with the median value of expression levels of XPG mRNA measured in a collection of tumor tissue in biopsy samples from human cancer patients;
c) classifying the patient in one of the 2 groups defined as “low level” when the expression level of XPG mRNA is lower than the median value of expression levels of XPG mRNA, and “high level” when the expression level of XPG mRNA is equal to or higher than the median value of expression levels of XPG mRNA; and
d) selecting said patient classified in the “high level” group for a chemotherapy treatment based on ET-743.

In another aspect, the invention relates to A screening method for selecting a human patient suffering from cancer for a treatment with ET-743, comprising the steps of:
a) determining the expression level of XPG protein in a biological sample of the patient;
b) recording the results of the determination in step (a) as negative expression (0), low expression (1+), moderate expression (2+), or high expression (3+); and
c) selecting said patient classified in the (2+) and (3+) groups for a chemotherapy treatment based on ET-743.

In another aspect, the invention relates to a screening method for selecting a human patient suffering from cancer for a treatment with ET-743, comprising the steps of:
a) determining the genotype of the Asp1104His SNP at locus rs17655 of the XPG gene in a biological sample of the patient;
b) classifying the patient in one of the 3 groups defined as “wild (W) type” genotype when a C nucleotide is present in at least one of the alleles of the SNP locus; “mutant (M)” genotype when a G nucleotide is present in both alleles of the SNP locus and the “heterozygous (H)” genotype when a C nucleotide is present in one allele and a G nucleotide in the other allele of the SNP locus;
c) selecting said patient classified in the “wild (W) type” or “heterozygous (H)” group for a chemotherapy treatment based on ET-743.

In yet another aspect, the invention relates to ET-743 for the treatment of cancer in human patients having tumours with high levels of XPG mRNA expression with respect to the median level of expression of XPG mRNA in a collection of biological samples.

In another aspect, the invention relates to ET-743 for the treatment of cancer in human patients having tumours with high levels of XPG protein expression.

In another aspect, the invention relates to ET-743 for the treatment of cancer in human patients having tumours with a “wild type” or “heterozygous (H)” genotype for the Asp1104His SNP at locus rs17655 of the XPG gene.

In a further aspect, the invention relates to the use of XPG protein or XPG mRNA as marker for the selection of human cancer patients to be effectively treated with ET-743.

In another aspect, the invention relates to the use of the Asp1104His SNP at locus rs17655 of the XPG gene as marker for the selection of human cancer patients to be effectively treated with ET-743.

### BRIEF DESCRIPTION OF THE FIGURES

FIG. 1. Kaplan-Meier plots of Progression Free Survival (PFS) and Overall Survival (OS) of the patients included in the study.

FIG. 2. Kaplan-Meier plot of PFS of patients according to their XPG mRNA expression levels in tumor samples.

FIG. 3. Kaplan-Meier plot of OS of patients according to their XPG mRNA expression levels in tumor samples.

FIG. 4. Kaplan-Meier plot of PFS of patients according to their XPG protein expression levels in tumor samples.

FIG. 5. Kaplan-Meier plot of OS of patients according to their XPG protein expression levels in tumor samples.

FIG. 6. Kaplan-Meier plot of PFS of patients according to their XPG mRNA and BRCA1 mRNA expression levels in tumor samples.

FIG. 7. Kaplan-Meier plot of OS of patients according to their XPG mRNA and BRCA1 mRNA expression levels in tumor samples.

FIG. 8. Kaplan-Meier plots of Progression Free Survival (PFS) and Overall Survival (OS) of the 168 patients according to the data in Table 5.

FIG. 9. Kaplan-Meier plot of PFS of patients according to their XPG SNP Asp1104His genotype in tumor samples.

FIG. 10. Kaplan-Meier plot of OS of patients according to their XPG SNP Asp1104His genotype in tumor samples.

FIG. 11. Kaplan-Meier plots of PFS of patients according to their XPG mRNA expression levels and XPG SNP Asp1104His genotype in tumor samples.

FIG. 12. Kaplan-Meier plots of OS of patients according to their XPG mRNA expression levels and XPG SNP Asp1104His genotype in tumor samples.

FIG. 13. Kaplan-Meier plots of PFS of patients according to their XPG protein expression levels and XPG SNP Asp1104His genotype in tumor samples.

FIG. 14. Kaplan-Meier plots of OS of patients according to their XPG protein expression levels and XPG SNP Asp1104His genotype in tumor samples.

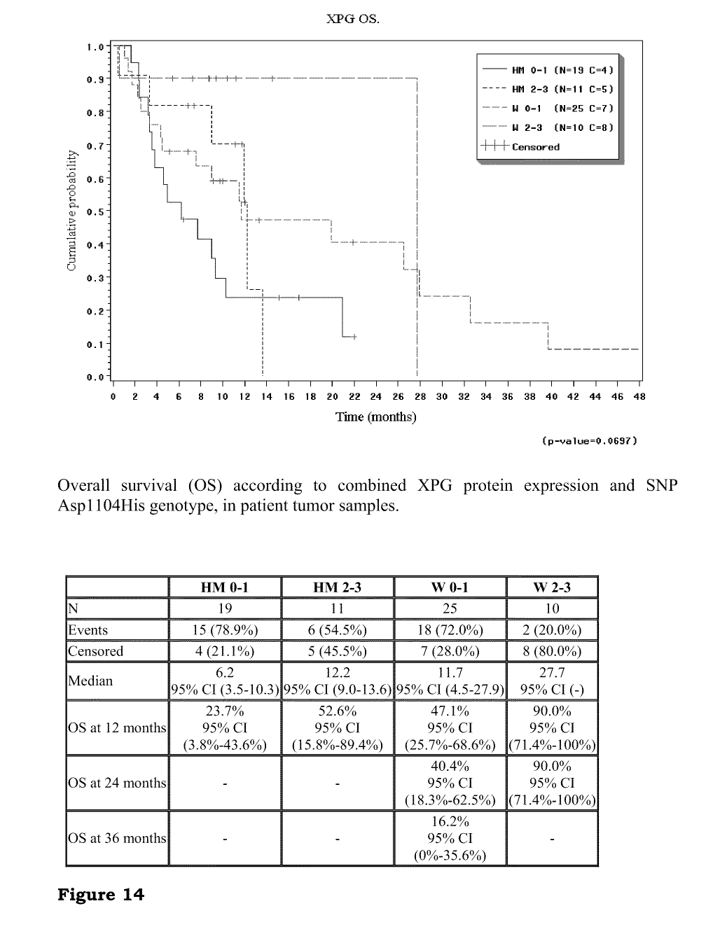

### DETAILED DESCRIPTION

### Method of Predicting the Clinical Response of a Cancer Patient to ET-743 Chemotherapy

The authors of the present invention have established that the tumour expression levels of XPG protein and XPG mRNA can play an important role in predicting differential chemotherapy sensitivity in human patients having cancer treated with ET-743. Specifically, as it is shown in example 1, human cancer patients having tumours with certain levels of expression of XPG, both determined as protein or as mRNA, are especially sensitive to the treatment with ET-743. In particular, the subdivision of a full cohort of patients in two equal subpopulations (“low” level of expression and “high” level of expression) according to their tumours XPG mRNA expression levels results in a significant increase of the efficacy of ET-743 in the subpopulation with increased expression:
from 10% to 19% of objective response (complete response (CR)+partial response (PR)),
from 36% to 56% of tumor control (CR+PR+minor response (MR)+stable disease (SD) 6 months),
from 2.5 to 7.1 months of median progression free survival (PFS),
from 29.5% to 52.1% of patients with PFS higher than 6 months (PFS6), and
from 9.3 to 19.1 months of median survival.
   Thus, in a first aspect, the invention relates to a method (hereinafter first method of the invention) for predicting the clinical response of a cancer patient to ET-743 chemotherapy comprising
a) determining the expression level of XPG mRNA in a biological sample of the patient before the ET-743 chemotherapy;
b) comparing the amount of expression of XPG mRNA in the biological sample with the median value of the expression of XPG mRNA measured in a collection of biological samples
   wherein an expression level of XPG mRNA equal to or higher than the median value of expression levels of XPG mRNA is indicative that the patient will show a positive response after treatment with ET-743.

The method of the invention allows the prediction of the clinical outcome of a patient. The expression “clinical outcome”, as used herein, relates to the determination of any parameter that can be useful in determining the evolution of a patient. The determination of the clinical outcome can be done by using any endpoint measurements used in oncology and known to the skilled practitioner. Useful endpoint parameters to describe the evolution of a disease include objective response, tumor control, progression free survival, progression free survival for longer than 6 months and median survival.

“Objective response”, as used in the present invention, describes the proportion of treated people in whom a complete or partial response is observed.

“Tumor control” relates to the proportion of treated people in whom complete response, partial response, minor response or stable disease 6 months is observed.

“Progression free survival” or PFS, as used herein, is defined as the time from start of treatment to the first measurement of cancer growth.

“Six-month progression free survival or PFS6” rate, as used herein, relates to the percentage of people wherein free of progression in the first six months after the initiation of the therapy.

“Median survival”, as used herein, relates to the time at which half of the patients enrolled in the study are still alive.

Step (a) of the first method of the invention requires the determination of the expression level of XPG mRNA in a sample. While all techniques of gene expression profiling (such as: RT-PCR, SAGE, DNA microarrays, or TaqMan®) are suitable for use in performing the foregoing aspects of the invention, the gene mRNA expression levels are often determined by reverse transcription polymerase chain reaction (RT-PCR). Preferably, the determination is carried out by quantitative (q-) RT-PCR, such as TaqMan®. The detection can be carried out in individual samples or in tissue microarrays.

In order to normalize the values of mRNA expression among the different samples, it is possible to compare the expression levels of the mRNA of interest in the test samples with the expression of a RNA mixture derived from multiple cell lines. Said RNA mixture (RNA controls used as calibrators) can be a commercial one, such as the universal human reference RNA (Stratagene) or a preparation made by pooling RNA preparations from all the samples to be analyzed. In a preferred embodiment, the quantification of gene expression is done by the comparative Ct method using an endogenous control RNA.

An “Endogenous control RNA” as used herein, relates to a RNA whose expression levels do not change or change only in limited amounts in tumor cells with respect to non-tumorigenic cells. Preferably, the “endogenous control RNA” are mRNA derived from housekeeping genes and which code for proteins which are constitutively expressed and carry out essential cellular functions. Preferred housekeeping genes for use in the present invention include β-2-microglobulin, ubiquitin, 18-S ribosomal protein, cyclophilin, GAPDH and actin. In a preferred embodiment, the control RNA is β-actin mRNA.

The present method can be applied to any type of biological sample from a patient, such as a biopsy sample, tissue, cell or fluid (serum, saliva, semen, sputum, cerebral spinal fluid (CSF), tears, mucus, sweat, milk, brain extracts and the like). For examination of tumor sensitivity to chemotherapy resistance, it is preferable to examine the tumor tissue. In a preferred embodiment, a portion of normal tissue from the patient from which the tumor is obtained, is also examined. Preferably this is done prior to the chemotherapy.

In performing the methods of the present invention, tumor cells are preferably isolated from the patient. Tumors or portions thereof are surgically resected from the patient or obtained by routine biopsy. RNA isolated from frozen or fresh samples is extracted from the cells by any of the methods typical in the art, for example, Sambrook, Fischer and Maniatis, Molecular Cloning, a laboratory manual, (2nd ed.), Cold Spring Harbor Laboratory Press, New York, (1989). Preferably, care is taken to avoid degradation of the RNA during the extraction process.

In a particular embodiment, the expression level is determined using mRNA obtained from a formalin-fixed, paraffin-embedded tissue sample. Other tissue samples are envisaged, such as fresh or frozen tissue from a biopsy or blood samples depending on their availability.

Fixed and paraffin-embedded tissue samples are preferred because they are broadly used storable or archival tissue samples in the field of oncology. mRNA may be isolated from an archival pathological sample or biopsy sample which is first deparaffinized. An exemplary deparaffinization method involves washing the paraffinized sample with an organic solvent, such as xylene, for example. Deparaffinized samples can be rehydrated with an aqueous solution of a lower alcohol. Suitable lower alcohols, for example include, methanol, ethanol, propanols, and butanols. Deparaffinized samples may be rehydrated with successive washes with lower alcoholic solutions of decreasing concentration, for example. Alternatively, the sample is simultaneously deparaffinized and rehydrated. The sample is then lysed and RNA is extracted from the sample.

Once the expression levels of XPG mRNA in the sample under study is determined, step (b) of the method of the invention comprises comparing the amount of expression of XPG mRNA in the biological sample with the median value of the expression of XPG mRNA measured in a collection of biological samples wherein an expression level of XPG mRNA equal to or higher than the median value of expression levels of XPG mRNA is indicative that the patient will show a positive response after treatment with ET-743.

Regarding the determination of XPG mRNA expression levels, the values for “low” and “high” levels of XPG mRNA expression are determined by comparison to reproducible standards (reference level) which may correspond to the median value of expression levels of XPG mRNA measured in a collection of tumor tissue in biopsy samples from cancer patients, previous to their treatment with ET-743. Once this median value is established, the level of this marker expressed in tumor tissues from patients can be compared with this median value, and thus be assigned a level of “low”, when XPG mRNA expression levels are lower than the median value of expression levels of XPG mRNA, or “high”, when XPG mRNA expression levels are equal to or higher than the median value of expression levels of XPG mRNA.

The XPG mRNA expression level is considered to be high when the levels in a sample from the subject under study are increased with respect to the median value by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%: by at least 85%, by at least 90%, by at least 95%, by at least 100%, by at least 110%, by at least 120%, by at least 130%, by at least 140% by at least 150%, or more. In a preferred embodiment, the XPG mRNA level are equal or higher than 1.5 is most preferred, in a still more preferred embodiment, the XPG mRNA level equal to or higher than 1.55.

The measure of relative gene expression is preferably made by using β-actin as an endogenous control, although other methods known in the art can be used, as long as relative levels of XPG mRNA can be assigned to the samples. Levels of XPG mRNA can be measured to obtain the relative level of XPG gene expression. Standard methods of measurement well known in the art can be used.

The collection of samples from which the reference level is derived will preferably be constituted from patients suffering from the same type of cancer. The collection may comprise, for example, samples from three, four, five, ten, 15, 20, 30, 40, 50 or more individuals. For example, the one described in the examples which is statistically representative was constituted with 160 samples from sarcoma patients. In any case it can contain a different number of samples.

In one embodiment relative gene expression quantification is calculated according to the comparative Ct method using β-actin as an endogenous control and commercial RNA controls as calibrators. Final results, are determined according to the formula 2-⁽^{^{Δ}}^{Ct sample-}^{^{Δ}}^{Ct calibrator}), where ΔC_{T} values of the calibrator and sample are determined by subtracting the C_{T} value of the target gene from the value of the β-actin gene.

The methods of the invention are suitable for predicting the clinical outcome of patients suffering from a wide variety of cancer types. By way of a non-limiting example, the inventions allows to predict the clinical response to chemotherapy with ET-743 of patients suffering from sarcoma, leiomyosarcoma, liposarcoma, osteosarcoma, ovarian cancer, breast cancer, melanoma, colorectal cancer, mesothelioma, renal cancer, endometrial cancer and lung cancer.

The authors of the present invention have also observed that an increase in the accuracy of the prediction of the clinical outcome after ET-743 therapy can be achieved by measuring also the expression levels of the BRCA1 mRNA. The comparison of the expression levels of XPG and BRCA1 mRNA in a biological sample with the median value of expression of XPG and BRCA1 mRNA measured in a collection of biological samples can be used for the determination of the clinical outcome since an expression level of XPG mRNA equal to or higher than the median value of expression levels of XPG mRNA and an expression level of BRCA1 mRNA lower than the median value of expression levels of BRCA1 mRNA is indicative that the patient will show a positive response after treatment with ET-743. In fact, table 4 shows that 63% of patient characterised by high XPG mRNA expression levels and low BRCA1 expression levels show progression free survival for 6 months when compared to a 56% when selected solely on the basis of XPG mRNA expression (Table 2).

Thus, in a preferred embodiment, the first method of the invention further comprises determining the expression levels of BRCA 1 mRNA in the biological sample with the median value of expression of XPG and BRCA1 mRNA measured in a collection of biological samples wherein an expression level of XPG mRNA equal to or higher than the median value of expression levels of XPG mRNA and an expression level of BRCA1 mRNA lower than the median value of expression levels of BRCA1 mRNA is indicative that the patient will show a positive response after treatment with ET-743.

Methods for the determination of the levels of BRCA1 mRNA are known to the skilled person and comprise essentially the same methods described above for the determination of the expression levels of XPG mRNA. In a preferred embodiment, the determination of the expression levels of BRCA1 mRNA is carried out using the procedures described in WO2006005602 which is hereby incorporated by reference.

The authors of the present invention have also observed that the combined analysis of the detection of the XPG mRNA expression levels and the genotype for the Asp1104His SNP at XPG gene allows a significant increase in the accuracy of the prediction of the clinical outcome of the ET-743 treatment in cancer patients. In particular, the inventors have observed that an expression level of XPG mRNA equal to or higher than the median value of expression levels of XPG mRNA and the presence of a C nucleotide in at least one of the alleles at the SNP locus is indicative that the patient will show a positive response after treatment with ET-743.

Thus, in a preferred embodiment, the first method of the invention based on the determination of the XPG mRNA levels and, optionally, of the BRCA1 levels, further comprises the determination of the genotype at locus rs17655 of the XPG gene wherein (i) an expression level of XPG mRNA equal to or higher than the median value of expression levels of XPG mRNA and (ii) the presence of a C nucleotide in at least one of the alleles at the SNP locus is indicative that the patient will show a positive response after treatment with ET-743 or wherein (i) an expression level of XPG mRNA equal to or higher than the median value of expression levels of XPG mRNA; (ii) an expression level of BRCA1 mRNA lower than the median value of expression levels of BRCA1 mRNA and (iii) the presence of a C nucleotide in at least one of the alleles at the SNP locus is indicative that the patient will show a positive response after treatment with ET-743.

It has also been found by the authors of the present invention that certain levels of XPG protein expression result in significant differences in the clinical outcome of the ET-743 treatment when comparing those patients having high protein expression levels (IHC (2+) and (3+)) in their tumors with those patient having low expression of XPG protein (IHC (0) and (1+)) in their tumors. In fact, high XPG protein expression levels produce a significant increase of the efficacy of ET-743:
from 13% to 24% of objective responses (CR+PR),
from 42% to 60% of tumor control (CR+PR+MR+SD 6 months),
from 3.7 to 7.1 months of median PFS,
from 39.9 to 55.6% of PFS6,
from 11.7 to 27.7 months of median survival, and
from 45.5% to 73.7% of one year survival rate.

Thus, in a further aspect, the invention relates to a method of predicting the clinical response of a cancer patient to ET-743 chemotherapy comprising
a) determining the expression level of XPG protein in a biological sample of the patient before the ET-743 chemotherapy;
b) recording the results of the determination of the expression levels of XPG protein as negative expression (0), low expression (1+), moderate expression (2+), or high expression (3+)
   wherein moderate or high expression levels of XPG protein is indicative that the patient will show a positive response after treatment with ET-743.

Step (a) requires the determination of the expression level of XPG protein in a sample from a patient before the ET-743. This step can be carried out using immunological techniques known to the skilled person such as e.g. ELISA, Western Blot or immunofluorescence. Western blot is based on the detection of proteins previously resolved by gel electrophoreses under denaturing conditions and immobilized on a membrane, generally nitrocellulose by the incubation with an antibody specific and a developing system (e.g. chemoluminiscent). The analysis by immunofluorescence requires the use of an antibody specific for the target protein for the analysis of the expression and subcellular localization by microscopy. Generally, the cells under study are previously fixed with paraformaldehyde and permeabilised with a non-ionic detergent. ELISA is based on the use of antigens or antibodies labelled with enzymes so that the conjugates formed between the target antigen and the labelled antibody results in the formation of enzymatically-active complexes. Since one of the components (the antigen or the labelled antibody) are immobilised on a support, the antibody-antigen complexes are immobilised on the support and thus, it can be detected by the addition of a substrate which is converted by the enzyme to a product which is detectable by, e.g. spectrophotometry or fluorometry. This technique does not allow the exact localisation of the target protein or the determination of its molecular weight but allows a very specific and highly sensitive detection of the target protein in a variety of biological samples (serum, plasma, tissue homogenates, postnuclear supernatants, ascites and the like).

In a preferred embodiment, the determination of the XPG protein levels is carried out by immunohistochemistry (IHC) analysis using thin sections of the biological sample immobilised on coated slides. The sections, when derived from a paraffin-embedded tissue samples, are deparaffinised and treated so as to retrieve the antigen. The detection can be carried out in individual samples or in tissue microarrays. When the expression analysis is carried out by IHC, the expression levels of XPG protein in a sample may be assigned to a semi-quantitative category of (0), (1+), (2+) or (3+) corresponding to, respectively, no color (that means no expression), low, medium and high staining, respectively, of the tumoral cells with the XPG specific antibody in biopsy samples from cancer patients. In another preferred embodiment, the semiquantitative categories (0), (1+), (2+) or (3+) are given based on the number of positive cells as follows: negative or (0) (no positive neoplastic cells), low or (1+) (1-15% positive neoplastic cells); medium or (2+) (>15-50% positive neoplastic cells) and high or (3+) (>50-100% positive neoplastic cells). This procedure, although is subjectively determined by the pathologist, is the standard method of measurement of IHC results, and well known in the art.

Any antibody or reagent known to specifically bind with high affinity to the target protein can be used for detecting the amount of target protein. It is preferred nevertheless the use of antibody, for example polyclonal sera, hybridoma supernatants or monoclonal antibodies, antibody fragments, Fv, Fab, Fab′ y F(ab′)2, ScFv, diabodies, triabodies, tetrabodies and humanised antibodies. Preferably, a monoclonal antibody is used. Said antibody or reagent specifically binding to the target protein will be labelled with a suitable marker, such as a fluorescent, chemoluminiscent, isotope marker, etc.

In a preferred embodiment, the determination of XPG protein expression levels can be carried out by constructing a tissue microarray (TMA) containing samples of multiple patients assembled, and determining the expression levels of XPG protein by immunohistochemistry techniques (ICH). Immunostaining intensity can be evaluated by two different pathologists and scored using uniform and clear cut-off criteria, in order to maintain the reproducibility of the method. Discrepancies can be resolved by simultaneous re-evaluation. Briefly, the result of immunostaining can be recorded as negative expression (0) versus positive expression, and low expression (1+) versus moderate (2+) and high (3+) expression, taking into account the expression in tumoral cells and the specific cut-off. As a general criterion, the cut-off is selected in order to facilitate reproducibility, and when possible, to translate biological events.

Therefore, the values for (0), (1+), (2+) or (3+) protein expression can be determined by the pathologist according to levels of staining of the IHC preparations. The (0), (1+), (2+) or (3+) values correspond to no color (that means no expression), low, medium and high staining, respectively, of the tumoral cells with the XPG specific antibody in biopsy samples from cancer patients, previous to their treatment with ET-743.

In step (b), the results of the determination in step (a) are recorded as negative expression (0), low expression (1+), moderate expression (2+), or high expression (3+) using the criteria mentioned above.In step (b), the XPG protein expression levels determined in step (a) are compared to a reference value. It will be understood that the expression of the expression level will depend on the methodology used for its determination. Thus, if the XPG protein levels are determined by ELISA or RIA, the “expression level” will be given as optical units or ppm. In this case, the “reference value” will be the expression levels of XPG protein in a cell population or biological sample obtained from healthy patients, or from a cell population obtained from a non tumor part of the tissue of the patient under study. More preferably, when a statistically sufficient population is gathered, the median value of the expression levels in all the samples of the population can be used as the reference value. Once the sample under study is assigned a given category, both moderate (2+) and high (3+) expression level of XPG protein are considered as “high expression” and thus, the patient wherein the sample is obtained is predicted to show a positive response after treatment with ET-743.

The authors of the present invention have also observed that an increased in the accuracy of the prediction of the clinical outcome can be achieved by also measuring the expression levels of the BRCA1 mRNA. Thus, in a preferred embodiment, the prediction method based on the levels of XPG protein further comprises the determination of the expression levels of BRCA1 mRNA wherein (i) an expression level of XPG protein higher than the reference value and (ii) moderate or high expression levels of XPG protein is indicative that the patient will show a positive response to the treatment with ET-743, wherein said moderate or high expression is determined using the scoring system mentioned above.

A expression level of XPG is considered as higher than a reference level when the fraction of cells expressing XPG above a threshold level is higher than 15% (the tissue sample shows a score of (2+) or (3+)).

In another aspect, the invention relates to a method of predicting the clinical response of a cancer patient to ET-743 chemotherapy comprising
a) determining the expression levels of XPG protein and BRCA1 mRNA in a biological sample of the patient;
b) comparing the amount of BRCA 1 mRNA relative to median value of expression of BRCA1 mRNA in a collection of biological samples
   wherein moderate or high expression levels of XPG protein and an expression level of BRCA1 mRNA lower than the median value of expression levels of BRCA 1 mRNA is indicative that the patient will show a positive response after treatment with ET-743.

The authors of the present invention have also observed that the simultaneous detection of the XPG protein expression levels and the genotype for the Asp1104His SNP at XPG gene allows a significant increase in the accuracy of the prediction of the clinical outcome of the ET-743 treatment in cancer patients. Thus, in a preferred embodiment, the methods for predicting the clinical outcome of a cancer patient treated with ET-743 based on the determination of the XPG protein levels and, optionally, of the BRCA1 levels, further comprise the determination of the genotype at locus rs17655 of the XPG gene wherein (i) moderate or high expression levels of XPG protein is higher than a reference expression level and (ii) the presence of a C nucleotide in at least one of the alleles at the SNP locus is indicative that the patient will show a positive response after treatment with ET-743 or wherein (i) moderate or high expression levels of XPG protein, (ii) an expression level of BRCA1 mRNA lower than the median value of expression levels of BRCA1 mRNA and (iii) the presence of a C nucleotide in at least one of the alleles at the SNP locus is indicative that the patient will show a positive response after treatment with ET-743.

The assignation of the XPG protein levels as higher than a reference value is given by recording the sample as moderate (2+) or high (3+) expression levels of XPG protein using the methods described above.

It has also been found by the inventors of the present application that the genotype for the Asp1104His SNP at XPG gene results in significant differences in the clinical outcome of the ET-743 treatment when comparing those patients having a wild type (W) genotype with those patients having a mutant (M) or heterozygous (H) genotype in their tumors. In particular, as shown in example 2, the subpopulation with the most favourable clinical outcome is defined by the presence of W genotype. The subpopulation having the M genotype show no benefit from ET-743 treatment and the subpopulation corresponding to the heterozygous genotype for the Asp1104His SNP show intermediate outcome, being the outcome defined as tumor control (CR+PR+MR+SD≧6), objective response and patients' survival (PFS and OS).

Thus, in a further aspect, the invention relates to a method of predicting the clinical response of a cancer patient to ET-743 chemotherapy comprising the step of determining the genotype of the Asp1104His SNP at locus rs17655 of the XPG gene in a biological sample of said patient before the ET-743 chemotherapy, wherein the presence of a C nucleotide in at least one of the alleles at the SNP locus is indicative that the patient will show a positive response after treatment with ET-743.

Genotyping of Asp1104His SNP in the XPG gene can be carried out by a great number of analytical procedures known in the art for the detection of SNP nucleotide variants, such as “allele amplification assay”, microarrays for SNP determination, direct sequencing, hybridisation probes, etc.

In a preferred embodiment, genotyping of Asp1104His SNP at XPG gene can be carried out by PCR amplification of the specific sequences from the extracted genomic DNA followed by RFLP analysis and confirmation of the genotype by direct sequencing across the amplified region containing the SNP locus as previously described (Le Morvan et al., Int. J. Cancer: 119, 1732-1735 (2006)). The wild type (W) genotype or Asp/Asp, is determined by the presence of the nucleotide C in both alleles at the SNP locus rs17655, corresponding to the position 3753 of the mRNA of XPG gene as described in NCBI accession number NM_{—}000123 (SEQ ID NO:1), coding for an Asp at the position 1104 of XPG protein. The variant/mutant (M) genotype is determined by the presence of a G nucleotide in both alleles at the SNP locus and the heterozygous (H) genotype is determined by the presence of C nucleotide in one allele and G in the other allele at the same locus.

In a particular embodiment, the SNP genotype is determined using genomic DNA obtained from a formalin-fixed, paraffin-embedded tissue sample. Other tissue samples are envisaged, such as fresh or frozen tissue from a biopsy, swaps, blood or other body fluids samples depending on their availability.

In addition, the authors of the present invention have also found that the combined analysis of the XPG Asp1104His SNP genotype and BRCA1 expression levels can further increase the accuracy in the prediction of clinical response to ET-743. In particular, it has been found that human patients having high levels of expression of XPG and low levels of expression of BRCA1 are more sensitive to the treatment with ET-743, and therefore in this subpopulation we have found an increase of the efficacy of ET-743.

Thus, in a preferred embodiment, the method of predicting a clinical response to ET-743 based on the Asp1104His SNP genotype in the XPG gene further comprises determining the expression level of BRCA1 mRNA in a biological sample of said patient before the ET-743 chemotherapy and comparing the amount of expression of BRCA1 mRNA in said biological sample with the median value of expression of BRCA1 mRNA measured in a collection of biological samples wherein (i) the presence of a C nucleotide in both alleles at the SNP locus rs17655 of the XPG gene and (ii) an expression level of BRCA1 mRNA lower than the median value of expression levels of BRCA1 mRNA is indicative that the patient will show a positive response after treatment with ET-743.

It has also been found that human patients having high levels of expression of XPG and a wild type genotype for the Asp1104His SNP at XPG gene are more sensitive to the treatment with ET-743. Thus, in a preferred embodiment, the method of predicting a clinical response to ET-743 based on the Asp1104His SNP genotype in the XPG gene further comprises determining the expression level of XPG mRNA in a biological sample of the patient before the ET-743 chemotherapy and comparing the amount of expression of XPG mRNA in said biological sample with the median value of the expression of XPG mRNA measured in a collection of biological samples wherein (i) the presence of a C nucleotide in both alleles at the SNP locus rs17655 of the XPG gene and (ii) an expression level of XPG mRNA equal to or higher than the median value of expression levels of XPG mRNA is indicative that the patient will show a positive response after treatment with ET-743.

In yet another embodiment, the method of predicting a clinical response to ET-743 based on the Asp1104His SNP genotype in the XPG gene further comprises determining the expression level of XPG protein in a biological sample of the patient before the ET-743 chemotherapy and recording the results of the determination under (a) as negative expression (0), low expression (1+), moderate expression (2+), or high expression (3+) wherein (i) the presence of a C nucleotide in both alleles at the SNP locus rs17655 of the XPG gene and (ii) a moderate or high expression level of XPG protein as defined above is indicative that the patient will show a positive response after treatment with ET-743.

In yet another embodiment, the method of predicting the clinical response of a cancer patient to ET-743 chemotherapy may include the determination of the genotype of the Asp1104His SNP at locus rs17655 of the XPG gene, the determination of the expression levels of XPG mRNA and the determination of the expression levels of BRCA1 in a biological sample wherein (i) the presence of a C nucleotide in both alleles at the SNP locus, (ii) an expression level of XPG mRNA equal to or higher than the median value of expression levels of XPG mRNA and (iii) an expression level of BRCA1 mRNA lower than the median value of expression levels of BRCA1 mRNA and is indicative that the patient will show a positive response after treatment with ET-743.

In yet another embodiment, the method of predicting the clinical response of a cancer patient to ET-743 chemotherapy may include the determination of the genotype of the Asp1104His SNP at locus rs17655 of the XPG gene, the determination of the expression levels of XPG protein and the determination of the expression levels of BRCA1 in a biological sample wherein (i) the presence of a C nucleotide in both alleles at the SNP locus rs17655 of the XPG gene, (ii) a moderate or high expression level of XPG protein and (iii) an expression level of BRCA1 mRNA lower than the median value of expression levels of BRCA1 mRNA is indicative that the patient will show a positive response after treatment with ET-743.

Method for Designing an Individual Chemotherapy for a Human Patient Suffering from Cancer

The authors of the present invention have also observed that chemotherapy based on ET-743 is particularly effective in patients with tumours having expression levels of XPG mRNA or protein equal to or above the median value of expression of XPG mRNA or protein, respectively, indicating that ET-743 would maintain its efficacy in those patients with poor response to Cisplatin or Doxorubicin due to the high expression levels of XPG.

Accordingly, in another aspect, the invention provides an in vitro method for designing an individual chemotherapy for a human patient suffering from cancer, comprising:
a) determining the expression level of XPG mRNA in a biological sample from said patient;
b) comparing the expression level of XPG mRNA obtained in a) with the value of the expression level of XPG mRNA measured in a collection of tumor tissue in biopsy samples from human cancer patients; and
c) selecting a chemotherapy treatment based on ET-743 when said XPG mRNA expression level is equal to or above the median value of expression levels of XPG mRNA.

It will be understood that the different elements of the second method of the invention (determination of XPG mRNA levels, determining a median value and the like) are carried out essentially as described for the first method of the invention.

Moreover, the authors of the present invention have also observed that the personalized chemotherapy based on ET-743 can also be decided on the basis of the combined detection of expression levels of BRCA1 and XPG mRNAs, so that the ET-743-based chemotherapy is selected in patients showing XPG mRNA expression levels equal to or above the median XPG mRNA expression levels and BRCA1 mRNA expression levels below the median BRCA1 mRNA expression levels. Accordingly, in a preferred embodiment, the in vitro method for designing an individual chemotherapy for a human patient suffering from cancer based on the determining the expression levels of XPG mRNA comprises comparing the expression levels of XPG and BRCA1 mRNAs obtained with median value of expression levels of XPG and BRCA1 mRNA measured in a collection of tumor tissue in biopsy samples from human cancer patients; and selecting a chemotherapy treatment based on ET-743 when said XPG mRNA expression level is equal to or above the median value of expression levels of XPG mRNA and BRCA1 mRNA expression level is below the median value of expression levels of BRCA1 mRNA.

The methods for designing an individual chemotherapy for a human patient suffering from cancer based on the levels of XPG mRNA and, optionally, including the determination of the expression levels of BRCA1 mRNA may be further improved by the simultaneous determination of the genotype of the Asp1104His SNP at locus rs17655 of the XPG in a biological sample from said patient.

Thus, in a preferred embodiment, the method for designing an individual chemotherapy based on the levels of XPG mRNA further comprises the steps of determining the genotype of the Asp1104His SNP at locus rs17655 of the XPG gene in a biological sample from said patient and selecting a chemotherapy treatment based on ET-743 when (i) XPG mRNA expression level is equal to or above the median value of expression levels of XPG mRNA and (ii) a C nucleotide is present in at least one of the alleles at the SNP locus.

In a further preferred embodiment, the method for designing an individual chemotherapy based on the levels of XPG mRNA further comprises the steps of determining the genotype of the Asp1104His SNP at locus rs17655 of the XPG gene in a biological sample from said patient and selecting a chemotherapy treatment based on ET-743 when (i) XPG mRNA expression level is equal to or above the median value of expression levels of XPG mRNA and (ii) a C nucleotide is present in both of the alleles at the SNP locus.

In yet another embodiment, the method for designing an individual chemotherapy based on the levels of XPG mRNA further comprises the step of determining the genotype of the Asp1104His SNP at locus rs17655 of the XPG gene in a biological sample from said patient and determining the expression levels of BRCA1 mRNA and selecting a chemotherapy treatment based on ET-743 when (i) XPG mRNA expression level is equal to or above the median value of expression levels of XPG mRNA; (ii) the BRCA1 mRNA expression level is below the median value of expression levels of the BRCA1 mRNA and (iii) a C nucleotide is present in at least one of the alleles at the SNP locus.

In a further preferred embodiment, the method for designing an individual chemotherapy based on the levels of XPG mRNA further comprises the step of determining the genotype of the Asp1104His SNP at locus rs17655 of the XPG gene in a biological sample from said patient and determining the expression levels of BRCA1 mRNA and selecting a chemotherapy treatment based on ET-743 when (i) XPG mRNA expression level is equal to or above the median value of expression levels of XPG mRNA; (ii) the BRCA1 mRNA expression level is below the median value of expression levels of the BRCA1 mRNA and (iii) a C nucleotide is present in both the alleles at the SNP locus.

In a further aspect, the invention provides an in vitro method for designing an individual chemotherapy for a human patient suffering from cancer, comprising:
a) determining the expression level of the XPG protein in a biological sample from said patient;
b) recording the results of the determination under (a) as negative expression (0), low expression (1+), moderate expression (2+), or high expression (3+); and
c) selecting a chemotherapy treatment based on ET-743 when said XPG protein expression level has a value of (2+) or (3+).

In a preferred embodiment, the determination of the XPG protein levels is carried out by immunohistochemistry analysis (ICH).

Moreover, the authors of the present invention have also observed that the method for designing a personalized chemotherapy based on ET-743 can also be decided on the basis of the simultaneous detection of expression levels of BRCA1 mRNA and XPG protein so that the ET-743-based chemotherapy is selected in patients showing XPG protein expression levels higher than a reference value (preferably, IHC scores of (2+) and (3+)), and BRCA1 mRNA expression levels below the median BRCA1 mRNA expression levels.

Accordingly, in a preferred embodiment, the in vitro method for designing an individual chemotherapy for a human patient suffering from cancer based on the determining the expression levels of XPG protein comprises determining the expression levels of XPG protein and BRCA1 mRNA in a biological sample from said patient; comparing the expression levels of XPG protein with a reference value, comparing the expression level of BRCA1 mRNA with the median value of expression of BRCA1 mRNA measured in a collection of biological samples and selecting a chemotherapy treatment based on ET-743 when said BRCA1 mRNA expression level is below the median value of expression levels of BRCA1 mRNA and the XPG protein expression has a value of (2+) or (3+).

The methods for designing an individual chemotherapy for a human patient suffering from cancer based on the levels of XPG protein and, optionally, considering the expression levels of BRCA1 mRNA, may be further improved by the simultaneous determination of the genotype of the Asp1104His SNP at locus rs17655 of the XPG in a biological sample from said patient.

Thus, in a preferred embodiment, the method for designing an individual chemotherapy based on the levels of XPG protein further comprises the steps of determining the genotype of the Asp1104His SNP at locus rs17655 of the XPG gene in a biological sample from said patient and selecting a chemotherapy treatment based on ET-743 wherein (i) the XPG protein expression level is (2+) or (3+) determined using the methods mentioned above and (ii) a C nucleotide is present in at least one of the alleles at the SNP locus.

In a more preferred embodiment, the method for designing an individual chemotherapy based on the levels of XPG protein further comprises the steps of determining the genotype of the Asp1104His SNP at locus rs17655 of the XPG gene in a biological sample from said patient and selecting a chemotherapy treatment based on ET-743 wherein (i) the XPG protein expression level is higher than a reference value (preferably when the expression score is (2+) or (3+)) and (ii) a C nucleotide is present in both of the alleles at the SNP locus.

In yet another embodiment, the method for designing an individual chemotherapy based on the levels of XPG protein further comprises the step of determining the BRCA1 mRNA expression levels and the genotype of the Asp1104His SNP at locus rs17655 of the XPG gene in a biological sample from said patient and selecting a chemotherapy treatment based on ET-743 when (i) the XPG protein expression level is (2+) or (3+) determined using the methods mentioned above, (ii) the BRCA1 mRNA expression level is below the median value of expression levels of the BRCA1 mRNA and (iii) a C nucleotide is present in at least one of the alleles at the SNP locus.

In a further preferred embodiment, the method for designing an individual chemotherapy based on the levels of XPG protein further comprises the step of determining the BRCA1 mRNA expression levels and the genotype of the Asp1104His SNP at locus rs17655 of the XPG gene in a biological sample from said patient and selecting a chemotherapy treatment based on ET-743 when (i) the XPG protein expression level is higher than a reference value (preferably when the expression score has a value of (2+) or (3+)); (ii) the BRCA1 mRNA expression level is below the median value of expression levels of the BRCA1 mRNA and (iii) a C nucleotide is present in both of the alleles at the SNP locus.

The authors of the present invention have also observed that chemotherapy based on ET-743 is particularly effective in patients showing a C nucleotide in at least one of the alleles of the SNP locus genotype of the Asp1104His SNP at locus rs17655 of the XPG gene. Thus, in another aspect, the invention relates to an in vitro method for designing an individual chemotherapy for a human patient suffering from cancer, comprising:
a) determining the genotype of the Asp1104His SNP at locus rs17655 of the XPG gene in a biological sample from said patient; and
b) selecting a chemotherapy treatment based on ET-743 when a C nucleotide is present in at least one of the alleles at the SNP locus.

The method for designing an individual chemotherapy according to the genotype in the XPG locus may further comprise the determination of additional parameters which enhance the accuracy of the determination. Thus, in a preferred embodiment, the method further comprises determining the expression level of BRCA1 mRNA in a biological sample of said patient before the ET-743 chemotherapy; comparing the amount of expression of BRCA1 mRNA in said biological sample with the median value of expression of BRCA1 mRNA measured in a collection of biological samples; and selecting a chemotherapy treatment based on ET-743 when (i) a C nucleotide is present in at least one of the alleles at the SNP locus and (ii) the BRCA1 mRNA expression level is below the median value of expression levels of BRCA1 mRNA .

In yet another embodiment, the method for designing an individual chemotherapy according to the genotype in the XPG locus further comprises determining the expression level of XPG mRNA in a biological sample of the patient before the ET-743 chemotherapy; and comparing the amount of expression of XPG mRNA in said biological sample with the median value of the expression of XPG mRNA measured in a collection of biological samples; and selecting a chemotherapy treatment based on ET-743 when (i) a C nucleotide is present in at least one of the alleles at the SNP locus and (ii) XPG mRNA expression level is equal to or above the median value of expression levels of XPG mRNA.

In yet another embodiment, the method for designing an individual chemotherapy according to the genotype in the XPG locus further comprises determining the expression level of the XPG protein in a biological sample from said patient, recording the results of the determination as negative expression (0), low expression (1+), moderate expression (2+), or high expression (3+) using the criteria previously mentioned and selecting a chemotherapy treatment based on ET-743 when (i) a C nucleotide is present inat least one of the alleles at the SNP locus and (ii) the XPG protein expression level has a value of (2+) or (3+).

In a further preferred embodiment, the invention relates to an in vitro method for designing an individual chemotherapy for a human patient suffering from cancer, comprising:
a) determining the genotype of the Asp1104His SNP at locus rs17655 of the XPG gene in a biological sample from said patient; and
b) selecting a chemotherapy treatment based on ET-743 when a C nucleotide is present in both of the alleles at the SNP locus.
   Method for Selecting a Human Patient Suffering from Cancer for a Treatment with ET-743

In a further aspect, the invention relates to a screening method for selecting a human patient suffering from cancer for a treatment with ET-743, comprising the steps:
a) determining the expression level of XPG mRNA in a biological sample of the patient;
b) comparing the expression level of XPG mRNA obtained in a) with the median value of expression levels of XPG mRNA measured in a collection of biological samples from human cancer patients;
c) classifying the patient in one of the 2 groups defined as “low level” when the expression level of XPG mRNA is lower than the median value of expression levels of XPG mRNA, and “high level” when the expression levels of XPG mRNA are equal or higher than the median value of expression levels of XPG mRNA; and
d) selecting said patient classified in the “high level” group for a chemotherapy treatment based on ET-743.

In yet another aspect, the invention relates to a screening method for selecting a human patient suffering from cancer for a treatment with ET-743, comprising the steps:
a) determining the expression level of XPG protein in a biological sample of the patient;
b) recording the results of the determination in step (a) as negative expression (0), low expression (1+), moderate expression (2+), or high expression (3+); and
c) selecting said patient classified in the (2+) and (3+) groups for a chemotherapy treatment based on ET-743.

In a preferred embodiment, in those methods of screening for selecting a human patient which involve the determination of the expression levels of XPG protein, the expression levels of this protein determined by immunohistochemistry analysis (IHC).

In another embodiment, the invention relates to a screening method for selecting a human patient suffering from cancer for a treatment with ET-743, comprising the steps of:
a) determining the genotype of the Asp1104His SNP at locus rs17655 of the XPG gene in a biological sample of the patient;
b) classifying the patient in one of the 3 groups defined as “wild (W) type” genotype when a C nucleotide is present in both alleles of the SNP locus; “mutant (M)” genotype when a G nucleotide is present in both alleles of the SNP locus and the “heterozygous (H)” genotype when a C nucleotide is present in one allele and a G nucleotide in the other allele of the SNP locus and
c) selecting a patient classified in the “wild (W) type” group or in the “heterozygous (H)” group for a chemotherapy treatment based on ET-743.

The methods of the invention for predicting the clinical response to the treatment with ET-743 in a patient suffering from cancer, the methods of the invention for selecting a patient for chemotherapy based on ET-743 or for designing an individual chemotherapy based on ET-743 for a patient can be applied to patients suffering from varied types of cancer, including, without limitation, sarcoma, leiomyosarcoma, liposarcoma, osteosarcoma, ovarian cancer, breast cancer, melanoma, colorectal cancer, mesothelioma, renal cancer, endometrial cancer and lung cancer; preferably soft tissue sarcomas, and most preferably leiomyosarcoma, liposarcoma or osteosarcoma.

As already described for the methods of the invention for predicting the clinical response of a patient suffering from cancer to the treatment with ET-743, the methods of the invention for selecting a patient for chemotherapy based on ET-743 or for designing an individual chemotherapy based on ET-743 for a patient can also be carried out in any type of sample from the patient, such as a biopsy sample, tissue, cell or fluid (serum, saliva, semen, sputum, cerebral spinal fluid (CSF), tears, mucus, sweat, milk, brain extracts and the like). For examination of tumor sensitivity to chemotherapy resistance, it is preferable to examine the tumor tissue. In a preferred embodiment, a portion of normal tissue from the patient from which the tumor is obtained, is also examined.

### Therapeutic Methods

The authors of the present invention have found that, surprisingly, the use of ET-743 in human cancer patients having certain expression levels of XPG, measured as XPG protein or as XPG mRNA, can lead to an increased antitumor efficacy in humans.

Thus, in another aspect, the invention is directed to the use of ET-743 for the treatment of cancer in human patients having high levels of XPG gene expression as detected by the mRNA expression. The invention also relates to a method of treating cancer in a human patient, comprising: determining the expression levels of XPG mRNA in a biological sample from said patient and treating the patient with ET-743 if said XPG mRNA expression levels are high.

Treatment of human cancer patients with a XPG mRNA level higher than 1 is preferred, an XPG mRNA level equal to or higher than 1.5 is most preferred, an XPG mRNA level equal to or higher than 1.55 being the most preferred.

The present invention also relates to the use of ET-743 for the treatment of cancer in human patients having high levels of expression of XPG protein. Preferably, the human cancer patients to be treated with ET-743 show a XPG protein expression levels higher than a reference value. Suitable reference values have been described previously. In a still more preferred embodiment, the XPG expression in a sample of the patient is recorded as negative expression (0), low expression (1+), moderate expression (2+), or high expression (3+) as described previously and whereby the patients to be treated are those showing expression levels higher than (1+) and more preferably equal to or higher than (2+). The invention also relates to a method of treating cancer in a human patient, comprising: determining the expression levels of XPG protein in a biological sample from said patient and treating the patient with ET-743 if said XPG protein expression levels are higher than a reference level.

The therapeutic methods of the invention based on the levels of XPG mRNA or protein may further comprise the determination of the levels of BRCA1 mRNA, wherein ET-743 is used in patients with high expression level of XPG mRNA and/or XPG protein is high and low expression levels of BRCA1 mRNA. Thus, the invention relates to ET-743 for use in the treatment of cancer in a patient if the expression level of XPG mRNA and/or XPG protein is high and if the expression levels of BRCA1 mRNA is low.

The invention also provides methods for the treatment of patients having a “wild type” genotype for the Asp1104His SNP at locus rs17655 of the XPG gene. In a preferred embodiment, ET-743 is used for the treatment of patients having a “wild type” genotype for the Asp1104His SNP at locus rs17655 of the XPG gene and ET-743 a low expression level of BRCA1. The invention also relates to a method for the treatment of a cancer patient with ET-743 wherein the patient has a “wild type” genotype for the Asp1104His SNP at locus rs17655 of the XPG gene and, optionally, if the patient shows low expression levels of ET-743.

The authors of the present invention evaluated previously if mRNA expression levels of DNA repair genes XPD, ERCC1 and BRCA1 might induce any differential sensitivity to ET-743 in cancer patients. It was found that XPD and ERCC1 do not induce any differential sensitivity to ET-743, for example in sarcoma patients, but it was surprisingly found that BRCA 1 has a high correlation to the clinical outcome of cancer patients treated with ET-743. Accordingly, in another aspect, the present invention also relates to the use of ET-743 for the treatment of cancer in human patients having high expression levels of XPG mRNA and low expression levels of BRCA1 mRNA. In yet another aspect, the present invention also relates to the use of ET-743 for the treatment of cancer in human patients having high expression levels of XPG protein and low expression levels of BRCA1 mRNA. Treatment of human cancer patients with a XPG mRNA level higher than 1 and a BRCA1 mRNA level lower than 3 is preferred; a XPG mRNA level equal to or higher than 1.5 and a BRCA 1 mRNA level equal to or lower than 2.5 is even more preferred; and a XPG mRNA level equal to or higher than 1.5 and a BRCA1 mRNA level equal to or lower than 2 is most preferred, and a XPG mRNA level equal to or higher than 1.55 and a BRCA1 mRNA level equal to or lower than 2.36 is the most preferred. In addition, treatment of human cancer patients with a score expression of XPG protein higher than (1+) and a BRCA1 mRNA level lower than 3 is preferred; a score expression of XPG protein equal to or higher than (2+) and a BRCA1 mRNA level equal to or lower than 2.5 is even more preferred; and a score expression of XPG protein equal to or higher than (2+) and a BRCA1 mRNA level equal to or lower than 2 is the most preferred.

The term “ET-743” is intended here to cover any pharmaceutically acceptable salt, ester, solvate, hydrate or any other compound which, upon administration to the patient is capable of providing (directly or indirectly) the compound as described herein. However, it will be appreciated that non-pharmaceutically acceptable salts also fall within the scope of the invention since these may be useful in the preparation of pharmaceutically acceptable salts. The preparation of salts and prodrugs and derivatives can be carried out by methods known in the art. ET-743 for use in accordance of this invention may be obtained as a natural product by isolation and purification from Ecteinascidia turbinata as described in available reference material. Alternatively, ET743 may be prepared by a hemisynthetic or synthetic process, see for example WO 00/69862 and WO 01/87895, which are both incorporated herein by reference.

ET-743 may be supplied and stored as a sterile lyophilized product, comprising ET-743 and an excipient in a formulation adequate for therapeutic use. In particular a formulation containing ET-743, sucrose and a phosphate salt buffered to an adequate pH is appropriate for the purposes of the present invention. In other suitable formulations, ET-743 in the form of a sterile lyophilized product is provided with mannitol and a phosphate salt buffered to an adequate pH. Further guidance on ET-743 formulations is given in WO 2006/046079, which is incorporated herein by reference in its entirety.

As single agent ET-743 has proven to induce long lasting objective remissions and tumor control in subsets of patients harbouring sarcomas relapsed to conventional therapy, ovarian cancer resistant or relapsed to Cisplatin-Paclitaxel and in breast cancer patients exposed to doxorubicin and to taxanes.

It is currently preferred to administer the ET-743 by infusion. The infusing step is typically repeated on a cyclic basis, which may be repeated as appropriate over for instance 1 to 20 cycles. The cycle includes a phase of infusing ET-743, and usually also a phase of not infusing ET-743. Typically the cycle is worked out in weeks, and thus the cycle normally comprises one or more weeks of an ET-743 infusion phase, and one or more weeks to complete the cycle. A cycle of 3 weeks is preferred, but alternatively it can be from 2 to 6 weeks. The infusion phase can itself be a single administration in each cycle of say 1 to 72 hours, more usually of about 1, 3 or 24 hours; or an infusion on a daily basis in the infusion phase of the cycle for preferably 1 to 5 hours, especially 1 or 3 hours; or an infusion on a weekly basis in the infusion phase of the cycle for preferably 1 to 3 hours, especially 2 or 3 hours. We currently prefer a single administration at the start of each cycle. Preferably the infusion time is about 1, 3 or 24 hour.

The dose will be selected according to the dosing schedule, having regard to the existing data on Dose Limiting Toxicity, on which see for example the above mentioned WO 00/69441 and WO 03/39571 patent specifications, and also see Kesteren, Ch. Van et al., 2003, Anti-Cancer Drugs, 14(7), 487-502. This article is also incorporated herein in full by specific reference.

Representative schedules and dosages are for example:

a) about 1.5 mg/ m² body surface area, administered as an intravenous infusion over 24 hours with a three week interval between cycles;

b) about 1.3 mg/m² body surface area, administered as an intravenous infusion over 3 hours with a three week interval between cycles;

c) about 0.580 mg/m² body surface area, administered weekly as an intravenous infusion over 3 hours during 3 weeks and one week rest.

As noted in the article by Kesteren, Ch. Van et al., the combination of ET-743 with dexamethasone gives unexpected advantages. It has a role in hepatic prophylaxis. We therefore prefer to administer dexamethasone to the patient, typically at around the time of infusing the ET-743. For example, we prefer to give dexamethasone on the day before ET-743, and/or the day after ET-743. The administration of dexamethasone can be extended, for example to more than one day following ET-743. In particular, we prefer to give dexamethasone at days—1, 2, 3 and 4 relative to a single administration of ET-743 on day 1 of a cycle.

In the use according to the present invention the compound ET-743 may be used with other drugs to provide a combination therapy. The other drugs may form part of the same composition, or be provided as a separate composition for administration at the same time or a different time. The identity of the other drug is not particularly limited, and suitable candidates include: a) drugs with antimitotic effects, especially those which target cytoskeletal elements, including microtubule modulators such as taxane drugs (such as paclitaxel, taxotere, docetaxel), podophylotoxins or vinca alkaloids (vincristine, vinblastine); b) antimetabolite drugs (such as 5-fluorouracil, cytarabine, gemcitabine, purine analogues such as pentostatin, methotrexate); c) alkylating agents or nitrogen mustards (such as nitrosoureas, cyclophosphamide or ifosphamide); d) drugs which target DNA such as the antracycline drugs adriamycin, doxorubicin, pharmorubicin or epirubicin; e) drugs which target topoisomerases such as etoposide; hormones and hormone agonists or antagonists such as estrogens, antiestrogens (tamoxifen and related compounds) and androgens, flutamide, leuprorelin, goserelin, cyprotrone or octreotide; g) drugs which target signal transduction in tumour cells including antibody derivatives such as herceptin; h) alkylating drugs such as platinum drugs (cis-platin, carbonplatin, oxaliplatin, paraplatin) or nitrosoureas; i) drugs potentially affecting metastasis of tumours such as matrix metalloproteinase inhibitors; j) gene therapy and antisense agents; k) antibody therapeutics; l) other bioactive compounds of marine origin, notably the didemnins such as aplidine; m) steroid analogues, in particular dexamethasone; n) anti-inflammatory drugs, including nonsteroidal agents (such as acetaminophen or ibuprofen) or steroids and their derivatives in particular dexamethasone; and o) anti-emetic drugs, including 5HT-3 inhibitors (such as palonisetron, gramisetron or ondasetron).

Depending on the type of tumor and the developmental stage of the disease, the treatments of the invention are useful in promoting tumor regression, in stopping tumor growth and/or in preventing metastasis. In particular, the method of the invention is suited for human patients, especially those who are relapsing or refractory to previous chemotherapy. First line therapy is also envisaged.

Although guidance for the dosage is given above, the correct dosage of the compound will vary according to the particular formulation, the mode of application, and the particular site, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

The use of ET-743 according to the invention is particularly preferred for the treatment of sarcoma, leiomyosarcoma, liposarcoma, osteosarcoma, ovarian cancer, breast cancer, melanoma, colorectal cancer, mesothelioma, renal cancer, endometrial cancer and lung cancer; preferably soft tissue sarcomas, and most preferably leiomyosarcoma, liposarcoma or osteosarcoma.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The following example further illustrates the invention. It should not be interpreted as a limitation of the scope of the invention.

### Examples

### Example 1

### Sample and Clinical Data Collection

In this study, 160 paraffin embedded tumor samples from sarcoma patients before the treatment with any chemotherapy agent have been evaluated.

The majority of patients were treated before with one or several chemotherapy agents and later they followed a treatment with ET-743. The dosage of intravenous infusion (IV) of ET-743 given to the different patients was within the range of 1.650-1.0 mg/m²; the schedules were 24 hours or 3 hour IV infusion with a three week interval between cycles; and the number of cycles ranged from 1 up to 44 cycles in some patients.

The clinical data from the patients was collected in the clinical data collection form and matched with the molecular data after completion of the mRNA and protein expression levels determination (Table 1).

Quantification of XPG mRNA Expression Levels

We examined XPG gene expression in formalin-fixed, paraffin-embedded tumor specimens from the 160 patients as previously described (Specht K. et al. Am J Pathol, 2001, 158, 419-429 and Krafft A E. et al. Mol Diagn. 1997, 3, 217-230). After standard tissue sample deparaffination using xylene and alcohols, samples were lysed in a tris-chloride, EDTA, sodium dodecyl sulphate (SDS) and proteinase K containing buffer. RNA was then extracted with phenol-chloroform-isoamyl alcohol followed by precipitation with isopropanol in the presence of glycogen and sodium acetate. RNA was resuspended in RNA storage solution (Ambion Inc; Austin TX, USA) and treated with DNAse I to avoid DNA contamination. cDNA was synthesized using M-MLV retrotranscriptase enzyme. Template cDNA was added to Taqman Universal Master Mix (AB; Applied Biosystems, Foster City, Calif., USA) in a 20-μl reaction with specific primers and probe for each gene. The primer and probe sets were designed using Primer Express 2.0 Software (AB). Quantification of gene expression was performed using the ABI Prism 7900HT Sequence Detection System (AB). The primers and 5′ labeled fluorescent reporter dye (6FAM) probe were as follows: β-actin: forward 5′ TGA GCG CGG CTA CAG CTT 3′ (SEQ ID NO:2), reverse 5′ TCC TTA ATG TCA CGC ACG ATT T 3′ (SEQ ID NO:3), probe 5′ ACC ACC ACG GCC GAG CGG 3′ (SEQ ID NO:4); XPG: forward 5′ GAA GCG CTG GAA GGG AAG AT 3′ (SEQ ID NO:5), reverse 5′ GAC TCC TTT AAG TGC TTG GTT TAA CC 3′ (SEQ ID NO:6), probe 5′ CTG GCT GTT GAT ATT AGC ATT 3′ (SEQ ID NO:7).

Relative gene expression quantification was calculated according to the comparative Ct method using β-actin as an endogenous control and commercial RNA controls (Stratagene, La Jolla, Calif.) as calibrators. Final results, were determined as follows: 2-^{(̂Ct sample-̂Ct calibrator}), where ΔC_{T} values of the calibrator and sample are determined by subtracting the C_{T} value of the target gene from the value of the β-actin gene. This was undertaken according to Technical Bulletin #2 recommended by the manufacturer (AB). In all experiments, only triplicates with a standard deviation (SD) of the Ct value <0.20 were accepted. In addition, for each sample analyzed, a retrotranscriptase minus control was run in the same plate to assure lack of genomic DNA contamination.

### Quantification of XPG Protein Expression Levels

Immunohistochemistry (ICH) analysis of XPG expression was performed in Tissue Micro-Arrays (TMA) containing tissue cylinders of tumor tissue from the patients studied. The TMA were constructed as follows: two 1.5-mm-diameter cylinders of tissue were taken from representative areas of each archival paraffin block and arrayed into a new recipient paraffin block with a custom-built precision instrument (Beecher Instruments, Silver Spring, Md.) (Kononen et al. (1998). Nat. Med. 1998, 4, 844-847). Areas chosen for the cylinder core had high tumor cellularity. In order to evaluate the most active part of each primary tumor, the invasive border of the tumor in large lesions and all tumor cells in smaller samples were selected. In addition, normal tissues (skin, tonsil, reactive lymphoid tissue) and three different cell lines with known cell-cycle alterations were placed adjacent to tumoral tissues to serve as internal controls and to ensure the quality of staining slides. Initial sections were stained for hematoxylin and eosin to verify the histopathological findings.

Characterization of XPG protein expression was performed by immunohistochemistry analysis (IHC), wherein three-micrometer tissue sections from the TMA blocks were sectioned and applied to special immunohistochemistry coated slides (Dako, Glostrup, Denmark). These slides were baked overnight in a 56° C. oven. Sections were deparaffinized in xylene for 20 minutes, rehydrated through a graded ethanol series and washed with phosphate-buffered saline. Antigen retrieval was achieved by a 2-minute heat treatment in a pressure cooker, containing 1 L of 10 mM sodium citrate buffer, pH 6.5, that was previously brought to the boil. Before staining, endogenous peroxidase activity was quenched with 1.5% hydrogen peroxide in methanol for 10 min. Immunohistochemical staining was performed on these sections using the XPG/ERCC5 Ab-1 (Clone 8H7), Mouse Monoclonal Antibody, (Thermo Scientific, Cat. #MS-674-P0), (dilution 1/100) for detection of XPG protein. After incubation, in the case of nuclear markers, immunodetection was performed with the LSAB Visualization System (Dako, Glostrup, Denmark) using 3,3′-diaminobenzidine chromogen as substrate, according to the manufacturer's instructions. In addition, whenever possible, cytoplasmic markers were visualized with the alkaline phosphatase anti-alkaline phosphatase system (APAAP system, Dako, Glostrup, Denmark), using neo-fuchsine chromogen as substrate to rule out the possibility of a role of endogenous melanin in the observed reactivity. All sections were counterstained with hematoxylin. Negative controls were obtained by omitting the primary antibody.

Immunostaining intensity was evaluated by two different pathologists and scored using uniform and clear cut-off criteria, in order to maintain the reproducibility of the method. Discrepancies were resolved by simultaneous re-evaluation. Briefly, the result of immunostaining was recorded as negative expression (0) versus positive expression, and low expression (1+) versus moderate (2+) and high (3+) expression, taking into account the expression in tumoral cells and the specific cut-off. As a general criterion, the cut-off is selected in order to facilitate reproducibility, and when possible, to translate biological events.

Therefore, the values for (0), (1+), (2+) or (3+) protein expression levels were determined by the pathologist according to levels of staining of the IHC preparations. The (0), (1+), (2+) or (3+) values correspond to no color (that means no expression), low, medium and high staining, respectively, of the tumoral cells with the XPG specific antibody in biopsy samples from cancer patients, previous to their treatment with ET-743. Each case was assigned to a semi-quantitative category based on the number of positive cells: negative or (0) (no positive neoplastic cells), low or (1+) (1-15% positive neoplastic cells); medium or (2+) (>15-50% positive neoplastic cells) and high or (3+) (>50-100% positive neoplastic cells). This procedure, although is subjectively determined by the pathologist, is the standard method of measurement of IHC results, and well known in the art are used.

Quantification of BRCA1 mRNA Expression Levels

BRCA1 mRNA expression levels were evaluated and quantified following the procedures already described in WO 2006/005602.

### Statistical Methods

SAS v8.2 (statistical software) was used for all the statistical analysis. The statistical techniques for univariate, bivariate and multivariate variables were chosen, according with the nature of variables that will be analysed, i.e. when the dependent variable is a temporal variable with censor status the Cox regression would be applied, when correlation between variables will be computed the Pearson and/or Spearman measures would be used. P-values below 0.05 will be considered statistically significant in all tests, when appropriate 95% confidence intervals will be presented too.

### Patient Cohort

A total of 160 paraffin embedded tumor samples providing evaluable results for mRNA or protein expression were analysed. Table 1 shows the most relevant clinical and molecular data of the 160 patients (CR: Complete Response; PR: Partial Response; MR: Minor Response; SD: Stable Disease; PD: Progressive Disease; OS: Overall survival; PFS: progression free survival). These samples came from sarcoma patients before being treated with a chemotherapy agent.

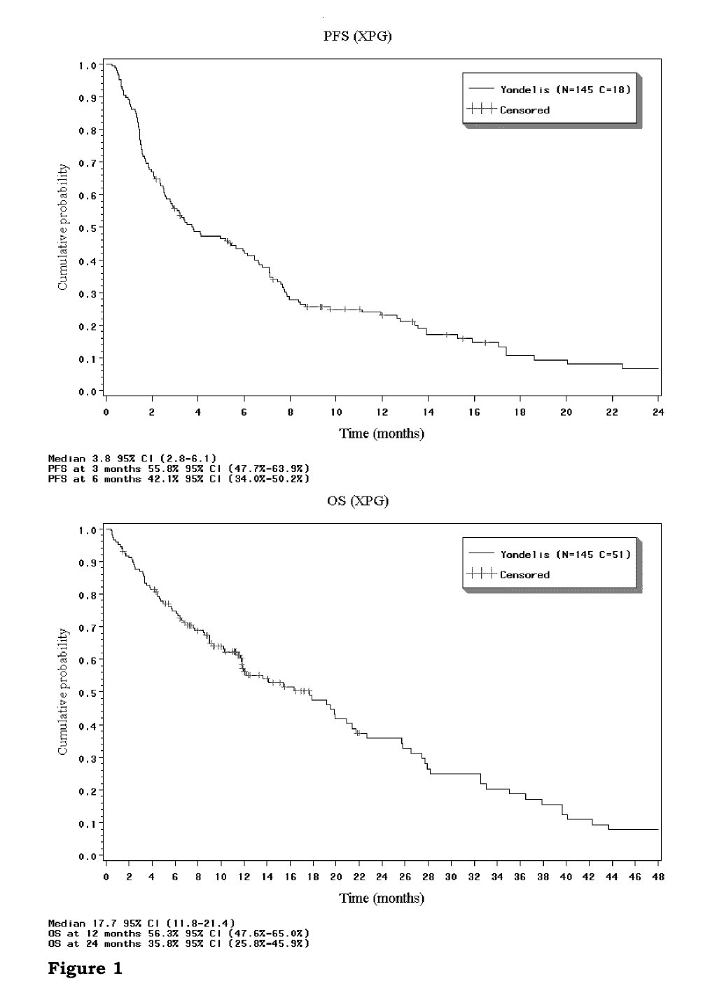

After treatment with ET-743, the overall response rate (RR) in 140 evaluable patients was 16.4% when considering Complete+Partial Responses ((1 CR+22 PR)/ 140 evaluable patients). In addition, 7.1% patients had Minor Responses (MR) (10 MR/140) and 32.9% (46/140 patients) had Stable Disease (SD). Tumor Control Rate ((CR+PR+MR+SD 6 mo) was achieved in 47.1% of the patients (66/140) and 58 patients (41.4%) achieved progression free survival 6 months (PFS6). The median duration of the response (CR+PR+MR) was 7.83 months (range 47.4 to 1.83 months) and 33 out of 46 SD reached the PFS6. Median survival was 10 months (0.4-65.9 months), although 51 patients are still censored. According to the Kaplan-Meier plots, the median progression free survival was 3.8 months and the PFS6 rate is 42.1% and the median survival is 17.7 months (FIG. 1).

TABLE 1
Clinical and molecular data of the 160 patients

| | | | | | PFS | OS | XPG | XPG | BRCA1 |
|---|---|---|---|---|---|---|---|---|---|
| Patient # | Age | Gender | Tumor Histology | Response | months | months | mRNA | protein | mRNA |
| 1 | 71 | Male | MFH | SD | 3.2 | 7.7 | | 0 | |
| 2 | 67 | Male | GIST | PD | 1.3 | 32.6 | 1.31 | | 2.84 |
| 3 | 77 | Male | Leiomyosarcoma | SD | 3.4 | 3.8 | 0.56 | 0 | 1.44 |
| 4 | 30 | Male | MPNST | PD | 0.5 | 1.1 | 1.27 | 0 | 3.95 |
| 5 | 42 | Male | MFH | PD | 1.6 | 2.3 | 0.98 | 1 | |
| 6 | 37 | Male | Liposarcoma | PR | 27.3 | 27.7 | 1.70 | 2 | |
| 7 | 40 | Male | Liposarcoma | PD | 1.7 | 3.3 | 1.09 | 0 | 2.71 |
| 8 | 49 | Female | MPNST | PD | 3.7 | 4.3 | 1.96 | 0 | 2.12 |
| 9 | 49 | Male | MFH | PD | 1.3 | 1.4 | 0.26 | 0 | 3.35 |
| 10 | 50 | Female | MFH | SD | 1.8 | 11.7 | 0.60 | 0 | 0.72 |
| 11 | 59 | Male | Liposarcoma | PD | 0.6 | 0.6 | | 2 | 3.37 |
| 12 | 37 | Female | MFH | PD | 1.7 | 1.7 | 0.91 | 0 | 0.74 |
| 13 | 36 | Female | Leiomyosarcoma | PD | 1.4 | 3.3 | 0.76 | 0 | |
| 14 | 28 | Female | Synovial Sarcoma | PD | 2.5 | 2.5 | 1.28 | 0 | 3.11 |
| 15 | 55 | Female | Leiomyosarcoma | PD | 1.4 | 4.5 | 0.47 | 0 | 1.40 |
| 16 | 34 | Female | Synovial Sarcoma | PD | 2.4 | 3.2 | 2.47 | 0 | 4.02 |
| 17 | 54 | Female | Liposarcoma | PD | 3.3 | 3.5 | 0.70 | 1 | 3.55 |
| 18 | 45 | Female | Synovial Sarcoma | SD | 13.6 | 26.5 | | 0 | 0.81 |
| 19 | 32 | Male | Synovial Sarcoma | SD | 17.4 | 20.9 | | 0 | |
| 20 | 64 | Female | Synovial Sarcoma | SD | 6.4 | 11.5 | 3.91 | 0 | 2.19 |
| 21 | 54 | Male | Leiomyosarcoma | PD | 1.1 | 11.8 | | 0 | |
| 22 | 52 | Male | Osteosarcoma | PD | 1.5 | 4.3 | | 0 | |
| 23 | 46 | Female | Other | PD | 2.5 | 6.1 | 1.51 | | 2.53 |
| 24 | 33 | Male | Osteosarcoma | PD | 2.8 | 6.6 | 0.53 | | 2.75 |
| 25 | 57 | Female | Myxoid Liposarcoma | PR | 22.4 | 28.2 | 1.96 | | 4.33 |
| 26 | 29 | Female | Other | PD | 1.3 | 11.9 | 2.01 | | 4.81 |
| 27 | 44 | Female | Synovial Sarcoma | PD | 0.7 | 58.4 | 0.66 | | — |
| 28 | 56 | Female | Leiomyosarcoma | PD | 1.3 | 21.4 | 0.31 | | 4.55 |
| 29 | 22 | Male | Osteosarcoma | PD | 1.5 | 2.9 | 1.40 | | — |
| 30 | 54 | Female | Liposarcoma | PD | 0.6 | 1.4 | 1.09 | | 8.96 |
| 31 | 18 | Female | Synovial Sarcoma | SD | 6.8 | 17.9 | 1.54 | | 9.22 |
| 32 | 54 | Male | Liposarcoma | PD | 1.1 | 10.2 | 1.59 | | 10.12 |
| 33 | 20 | Male | Other | SD | 3.2 | 40.1 | 1.66 | | 1.38 |
| 34 | 64 | Male | Other | SD | 7.7 | 25.7 | 0.85 | | 1.54 |
| 35 | 35 | Female | Ewing Sarcoma | PD | 1.0 | 17.7 | 1.49 | | 2.19 |
| 36 | 43 | Male | Other | PD | 1.0 | 22.7 | 2.21 | | 2.37 |
| 37 | 26 | Female | Synovial Sarcoma | PR | 3.1 | 19.1 | 2.71 | | 3.68 |
| 38 | 38 | Male | Other | SD | 4.1 | 15.4 | 3.17 | | 2.03 |
| 39 | 34 | Female | Synovial Sarcoma | PD | 2.1 | 4.7 | 2.33 | | 4.14 |
| 40 | 30 | Male | Other | SD | 7.8 | 37.9 | 2.85 | | — |
| 41 | 38 | Female | Uterine | PD | 1.5 | 6.7 | 0.75 | | 0.92 |
| | | | Leiomyosarcoma | | | | | | |
| 42 | 40 | Female | MPNST | PD | 2.1 | 2.4 | 1.35 | | 1.13 |
| 43 | 19 | Male | Ewing Sarcoma | PD | 0.7 | 4.5 | 0.61 | | 3.92 |
| 44 | 28 | Male | Other | PD | 1.4 | 5.4 | 0.51 | | 4.53 |
| 45 | 28 | Female | Other | SD | 7.5 | 21.7 | 1.98 | | |
| 46 | 35 | Male | Other | PD | 0.7 | 3.2 | 2.43 | | 2.81 |
| 47 | 41 | Female | Uterine | PR | 17.4 | 43.7 | 3.02 | | 0.58 |
| | | | Leiomyosarcoma | | | | | | |
| 48 | 21 | Male | Osteosarcoma | PD | 1.5 | 6.4 | 2.99 | | 5.66 |
| 49 | 42 | Female | Osteosarcoma | SD | 7.1 | 14.1 | 3.65 | | 8.23 |
| 50 | 66 | Female | Other | MR | 3.8 | 5.7 | 3.86 | | 2.12 |
| 51 | 60 | Female | Other | MR | 9.7 | 25.8 | 2.26 | | 2.32 |
| 52 | 37 | Female | Other | PD | 0.7 | 1.2 | 1.76 | | 5.13 |
| 53 | 38 | Female | Other | MR | 2.5 | 16.4 | 0.80 | | |
| 54 | 59 | Male | Myxoid Liposarcoma | PR | 16.5 | 16.5 | | 2 | |
| 55 | 65 | Male | Myxoid Liposarcoma | PR | 3.0 | 14.5 | | 2 | |
| 56 | 35 | Female | Leiomyosarcoma | PR | 8.7 | 11.8 | 1.69 | 0 | 0.64 |
| 57 | 52 | Male | Myxoid Liposarcoma | PR | 15.5 | 15.5 | 3.79 | | 5.92 |
| 58 | 52 | Male | Myxoid Liposarcoma | PR | 15.5 | 15.5 | 4.41 | | 3.69 |
| 59 | 56 | Male | Myxoid Liposarcoma | MR | 6.6 | 15.1 | 0.79 | 0 | 1.59 |
| 60 | 64 | Female | Uterine | PR | 5.9 | 7.1 | 1.04 | 0 | 0.83 |
| | | | Leiomyosarcoma | | | | | | |
| 61 | 56 | Male | Other | PD | 0.5 | 0.5 | 1.59 | 0 | 3.13 |
| 62 | 20 | Female | sacro-iliacal right | PD | 1.5 | 1.5 | | 0 | |
| 63 | 70 | Female | Leiomyosarcoma | PD | 0.5 | 0.5 | 1.87 | 0 | 0.92 |
| 64 | 70 | Female | Leiomyosarcoma | SD | 8.7 | 8.7 | 0.41 | | 2.35 |
| 65 | 54 | Male | Liposarcoma | SD | 3.2 | 11.8 | 1.14 | 0 | 2.73 |
| 66 | 51 | Female | Myxoid Liposarcoma | PR | 7.1 | 7.1 | 1.15 | 1 | 0.80 |
| 67 | 53 | Female | Uterine | PD | 2.3 | 11.3 | 0.43 | 0 | 0.38 |
| | | | Leiomyosarcoma | | | | | | |
| 68 | 74 | Female | Myxoid Liposarcoma | MR | 5.3 | 12.4 | 2.45 | | 0.94 |
| 69 | 54 | Male | Other | SD | 2.2 | 12.5 | 1.67 | 2 | 0.58 |
| 70 | 60 | Female | Leiomyosarcoma | PD | 2.3 | 12.1 | 1.11 | 1 | 1.55 |
| 71 | 40 | Male | Liposarcoma | MR | 14.8 | 17.2 | 1.28 | | 2.29 |
| 72 | 25 | Male | Other | PR | 7.3 | 14.0 | 2.23 | | 2.34 |
| 73 | 42 | Male | Other | PD | 1.7 | 8.0 | 1.08 | | 0.37 |
| 74 | 56 | Male | Myxoid Liposarcoma | MR | 6.13 | 11.6 | 1.07 | | |
| 75 | | | Osteosarcoma | | | | | 0 | 3.40 |
| 76 | | | Leiomyosarcoma | | | | | 0 | 3.54 |
| 77 | | | Leiomyosarcoma | | | | | 0 | 4.86 |
| 78 | | Male | Osteosarcoma | | | | 0.99 | | 2.38 |
| 79 | 69 | Male | Myxoid Liposarcoma | PR | 11.2 | 11.1 | 1.47 | 2 | 2.19 |
| 80 | 53 | Male | Myxoid Liposarcoma | PD | 1.5 | 9.4 | 3.08 | | 3.69 |
| 81 | 58 | Male | Myxoid Liposarcoma | PD | 3.4 | 13.6 | 2.17 | 2 | 2.73 |
| 82 | 59 | Female | Other | PD | 1.5 | 2.3 | 1.05 | 0 | 1.74 |
| 83 | 57 | Female | Leiomyosarcoma | PR | 9.7 | 9.7 | | 0 | 0.50 |
| 84 | 70 | Male | Liposarcoma | SD | 10.4 | 10.4 | 4.51 | 2 | |
| 85 | 43 | Female | Myxoid Liposarcoma | SD | 8.0 | 8.8 | 1.27 | 2 | 2.20 |
| 86 | | | | PD | 1.5 | 1.6 | 2.92 | 1 | 3.68 |
| 87 | 45 | Female | Uterine | PD | 2.5 | 3.9 | 0.89 | | 3.66 |
| | | | Leiomyosarcoma | | | | | | |
| 88 | 47 | Female | Uterine | SD | 15.9 | 36.5 | | 0 | |
| | | | Leiomyosarcoma | | | | | | |
| 89 | 61 | Male | MFH | SD | 42.0 | 42.3 | | 2 | |
| 90 | 18 | Female | Other | PR | 20.1 | 52.7 | 0.71 | | 0.65 |
| 91 | 42 | Male | Other | PD | 5.4 | 19.8 | — | 1 | |
| 92 | | Female | Liposarcoma | | | | 2.53 | | 9.34 |
| 93 | 50 | Female | | PR | 13.4 | 27.4 | | 1 | 17.13 |
| 94 | 45 | Male | | SD | 7.9 | 35.1 | | 0 | 1.98 |
| 95 | 35 | Female | | MR | 29.0 | 64.1 | | 0 | |
| 96 | | | | | | | | 0 | |
| 97 | 60 | Male | Myxoid Liposarcoma | SD | 9.3 | 9.3 | 3.10 | 2 | 1.62 |
| 98 | 55 | Female | Leiomyosarcoma | SD | 7.8 | 10.0 | 0.68 | 0 | 2.62 |
| 99 | 72 | Female | Leiomyosarcoma | SD | 6.7 | 10.0 | 1.30 | 0 | 2.56 |
| 100 | 62 | Male | Liposarcoma | SD | 11.0 | 11.0 | 5.68 | | 1.25 |
| 101 | 55 | Male | Liposarcoma | PD | 2.0 | 2.4 | 1.71 | 0 | 3.42 |
| 102 | 47 | Male | Myxoid Liposarcoma | PR | 15.3 | 22.0 | | 0 | |
| 103 | 74 | Male | Other | SD | 7.3 | 7.2 | 7.54 | 2 | 2.96 |
| 104 | 53 | Female | GIST | PD | 2.6 | 6.9 | 1.19 | 0 | 4.85 |
| 105 | 61 | Female | Uterine | PD | 3.5 | 6.8 | 0.91 | 2 | 2.85 |
| | | | Leiomyosarcoma | | | | | | |
| 106 | 69 | Male | Myxoid Liposarcoma | PR | 7.1 | 7.4 | 8.15 | 2 | 3.72 |
| 107 | 54 | Female | Uterine | | | | 0.71 | 1 | 3.35 |
| | | | Leiomyosarcoma | | | | | | |
| 108 | 70 | Male | Leiomyosarcoma | SD | 8.4 | 33.0 | 4.29 | 0 | 2.16 |
| 109 | 48 | Male | Myxoid Liposarcoma | SD | 2.1 | 3.3 | 1.62 | 2 | 0.77 |
| 110 | 62 | Female | Synovial Sarcoma | SD | 4.1 | 12.0 | 4.30 | 2 | |
| 111 | 70 | Male | Rabdomiosarcoma | | 0.4 | 0.4 | | 2 | |
| 112 | 47 | Male | Liposarcoma | PD | 1.6 | 9.0 | 1.40 | 3 | 0.62 |
| 113 | 65 | Male | GIST | PD | 1.4 | 19.9 | 1.86 | 0 | 2.82 |
| 114 | 49 | Female | Leiomyosarcoma | SD | 7.7 | 9.0 | 1.36 | 0 | 1.30 |
| 115 | 54 | Female | Leiomyosarcoma | SD | 3.8 | 8.5 | | 0 | |
| 116 | 23 | Female | Synovial Sarcoma | SD | 18.6 | 53.5 | 2.93 | 0 | 1.50 |
| 117 | 39 | Male | Other | | 0.9 | 0.9 | | 0 | |
| 118 | 25 | Female | Other | PR | 17.1 | 27.9 | 2.53 | 0 | |
| 119 | 63 | Male | MFH | SD | 29.7 | 32.6 | 1.32 | 0 | 0.92 |
| 120 | 33 | Male | Other | PD | 2.9 | 4.6 | 1.55 | 0 | |
| 121 | 35 | Male | Other | SD | 13.9 | 39.6 | | 0 | |
| 122 | 65 | Female | Leiomyosarcoma | PD | 1.9 | 7.6 | 0.28 | 0 | 0.84 |
| 123 | 33 | Male | GIST | | 5.0 | 5.0 | 2.22 | 0 | 1.26 |
| 124 | 70 | Female | Leiomyosarcoma | | 1.8 | 9.3 | 1.15 | 1 | 4.09 |
| 125 | 43 | Female | Leiomyosarcoma | SD | 5.6 | 16.9 | 0.53 | 0 | 0.17 |
| 126 | 59 | Female | Liposarcoma | SD | 12.0 | 12.0 | 2.84 | 2 | 1.60 |
| 127 | 47 | Female | Leiomyosarcoma | PD | 1.6 | 11.8 | 0.27 | 2 | 1.43 |
| 128 | 62 | Female | Leiomyosarcoma | PD | 2.8 | 6.2 | 0.37 | 0 | 0.29 |
| 129 | 57 | Female | Liposarcoma | SD | 5.2 | 10.3 | | 0 | |
| 130 | 52 | Male | Liposarcoma | PR | 6.0 | 8.7 | 1.29 | 3 | 0.31 |
| 131 | 73 | Male | Liposarcoma | SD | 13.3 | 13.3 | 3.31 | 0 | 2.63 |
| 132 | | | Leiomyosarcoma | | | | 0.93 | | |
| 133 | | | Leiomyosarcoma | | | | 0.53 | | |
| 134 | 9 | Male | Ewing Sarcoma | | | | 1.84 | | 3.20 |
| 135 | | | Ewing Sarcoma | | 1.0 | 2.0 | 5.46 | | 1.71 |
| 136 | | | Liposarcoma | | | | 2.47 | | 2.66 |
| 137 | 74 | Male | Liposarcoma | PD | 0.8 | 4.2 | | 0 | |
| 138 | 74 | Male | Liposarcoma | PD | 0.8 | 4.2 | | 2 | |
| 139 | 72 | Male | Liposarcoma | CR | 12.8 | 17.6 | | 0 | |
| 140 | 47 | Male | | PD | 0.3 | 0.7 | 5.54 | | |
| 141 | 70 | Male | | SD | 11.9 | 19.5 | 6.15 | | |
| 142 | 56 | Male | | PR | 47.4 | 65.9 | 1.55 | | |
| 143 | 54 | Male | Leiomyosarcoma | SD | 8.4 | 9.0 | 0.53 | 0 | 0.62 |
| 144 | 57 | Female | Leiomyosarcoma | PD | 0.7 | 8.6 | 0.29 | | 0.65 |
| 145 | 56 | Female | Uterine | MR | 6.4 | 6.40 | 0.94 | 0 | 4.58 |
| | | | Leiomyosarcoma | | | | | | |
| 146 | 54 | Female | Uterine | SD | 7.6 | 11.9 | 0.30 | | |
| | | | Leiomyosarcoma | | | | | | |
| 147 | 42 | Female | Uterine | PD | 2.9 | 5.2 | | 0 | |
| | | | Leiomyosarcoma | | | | | | |
| 148 | 36 | Female | Myxoid Liposarcoma | SD | 7.1 | 11.2 | 4.91 | 3 | 2.36 |
| 149 | 34 | Male | Myxoid Liposarcoma | SD | 12.6 | 21.8 | 3.03 | 1 | |
| 150 | 48 | Female | Uterine | PD | 1.4 | 5.8 | 0.65 | | |
| | | | Leiomyosarcoma | | | | | | |
| 151 | 52 | Male | Myxoid Liposarcoma | PD | 1.5 | 12.2 | 2.40 | 3 | 0.35 |
| 152 | 59 | Female | Uterine | PR | 5.3 | 11.5 | 0.88 | | 1.19 |
| | | | Leiomyosarcoma | | | | | | |
| 153 | 28 | Female | Uterine | SD | 2.6 | 9.1 | 1.56 | 0 | 3.11 |
| | | | Leiomyosarcoma | | | | | | |
| 154 | 70 | Male | Myxoid Liposarcoma | SD | 9.4 | 9.4 | 8.68 | 3 | 2.84 |
| 155 | 35 | Female | Chondrosarcoma | | | | 3.94 | 2 | 1.67 |
| 156 | 35 | Male | Other | SD | 13.9 | 39.6 | 1.56 | 0 | |
| 157 | 38 | Male | Myxoid Liposarcoma | MR | 5.4 | 5.4 | 8.26 | 3 | 7.00 |
| 158 | 15 | Male | Ewing Sarcoma | | | | 3.45 | | 6.75 |
| 159 | | | | | | | 7.23 | | |
| 160 | | Male | | | | | 1.43 | | |

Correlation of XPG mRNA Expression Levels and ET-743 Treatment Outcome.

The amount of XPG mRNA relative to the 13-actin (internal control) was determined in 116 samples. This amount was ranging from 0.26 to 8.68, a 33.4-fold difference from the minimum to the maximum value found. The median expression value was 1.55.

The association between the expression level of XPG mRNA with the clinical outcome of the patients treated with ET-743 is shown in Table 2.

TABLE 2

| | XPG Expression Levels | | |
|---|---|---|---|
| Parameter | XPG < 1.55 | XPG ≧ 1.55 | p-Value |
| CR + PR | 6/59 (10%) | 11/57 (19%) | 0.1957 |
| CR + PR + MR + SD ≧ 6 | 21/59 (36%) | 32/57 (56%) | 0.0399 |
| PFS ≧ 6 Months rate | 18/60 (30%) | 28/59 (47.5%) | 0.0609 |
| Median PFS (KM) | 2.5 m | 7.1 m | 0.0021 |
| PFS6 (KM) | 29.5% | 52.1% | 0.0107 |
| Median Survival (KM) | 9.3 m | 19.1 m | 0.2367 |

KM: according to Kaplan-Meier plot

Table 2 shows that the rates for Objective Response and Tumor Control are higher in patients having expression values of XPG mRNA above the median value (1.55) of the cohort. The Objective Response of these patients having high levels of expression of XPG mRNA is 19% and the Tumor Control is 56%, compared with those with low expression levels of XPG mRNA who showed an Objective Response of 10% and a Tumor Control of 36%.

Similarly, the probability of reaching PFS6 is statistically significant higher in those patients having high XPG mRNA expression. In fact, according to KM plots 52.1% patients having high XPG mRNA expression reached the PFS6 endpoint meanwhile only 29.5% of the patient with low expression reached PFS6. The fact that the correlation is significant with clinical response and PFS6 indicate that XPG mRNA expression level is a marker for the treatment with ET-743 and not a marker of tumor aggressiveness.

This means that, subdivision of the full cohort of patients in two equal subpopulations according to the XPG mRNA expression produces an increase of the efficacy of ET-743 in the target subpopulation from 10% ( 6/59) to 19% ( 11/57) in objective response (1.9 fold increase) and from 36% ( 21/59) to 56% ( 32/57) in tumor control rate (1.6 fold increase).

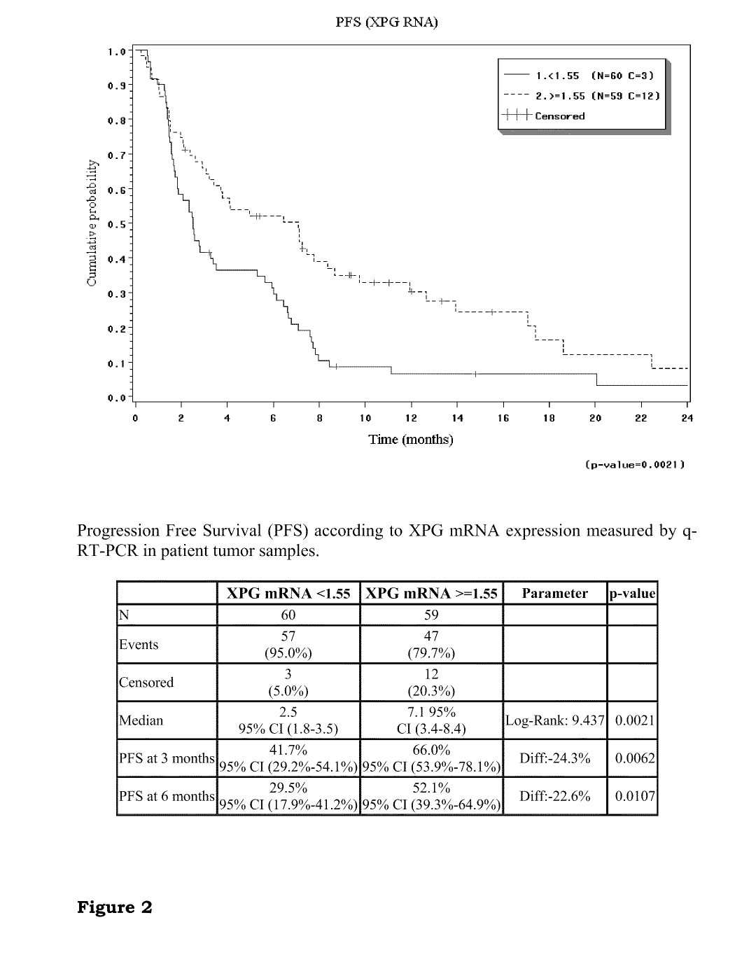

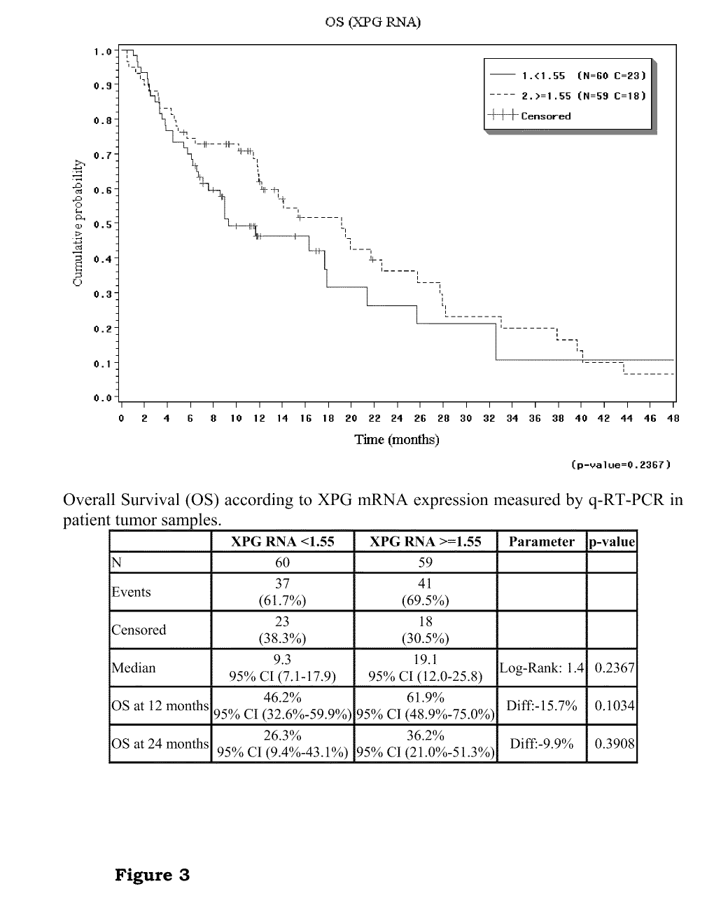

The Kaplan-Meier plots of FIGS. 2 and 3 show a statistically significant difference [p=0.0021] in PFS and a trend [p=0.2367] in survival, respectively, on those patients having a XPG expression under the median (1.55). The median survival was 19.1 months for high expressers and 9.3 for low expression patients. In addition, the median PFS was 7.1 months for high expression patients and 2.5 months for low expression patients, and the percentage of patients with PFS6 was 29.5% and 52.1%, respectively. These differences are statistically significant [p=0.0021] and [p=0.0107], respectively. Finally, the survival at 12 months was 61.9% for those patients having high XPG mRNA expression and 46.2% for those patients having low XPG mRNA expression [p=0.1034].

### Correlation of XPG Protein Expression Levels and ET-743 Treatment Outcome.

The amount of XPG protein was determined in 92 samples of paraffin embedded tumor tissue from sarcoma patients treated with ET-743. The score of expression was determined as 0 (no expression of XPG protein), 1+(low expression), 2+(moderate expression) and 3+(high expression). The number of samples in each expression scoring group was 58 (60.0%), 9 (9.5%), 20 (21.7%) and 5 (5.4%), respectively.

In order to test the association between the expression levels of XPG protein with the clinical outcome of the patients treated with ET-743, the patients were grouped in two subpopulations: the low expressers those having low or no expression of XPG proteins (Scores 0 and 1+: 67 patients) and the high expressers having intermediate and high expression (scores 2+and 3+: 25 patients). The correlation of clinical outcome in these two subpopulations is shown in Table 3.

TABLE 3

| | XPG Protein Levels | | |
|---|---|---|---|
| Parameter | XPG 0 + 1 | XPG 2 + 3 | p-Value |
| CR + PR | 8/64 (13%) | 6/25 (24%) | 0.2041 |
| CR + PR + MR + SD ≧ 6 | 27/64 (42%) | 15/25 (60%) | 0.1597 |
| PFS ≧ 6 Months | 26/64 (39%) | 13/25 (50%) | 0.3566 |
| Median PFS (KM) | 3.7 m | 7.1 m | 0.0411 |
| PFS6 (KM) | 39.9% | 55.6% | 0.1842 |
| Median Survival (KM) | 11.7 m | 27.7 m | 0.107 |

Table 3 shows that the rates for Objective Response and Tumor Control are higher in patients having expression scores of XPG protein of 2+ and 3+. The Objective Response of these patients having an expression score of 2+ and 3+ is 24% and the Tumor Control is 60%, compared with those with an expression score of 0 and 1+ who showed an Objective Response of 13% and a Tumor Control of 42%.

Similarly, the probability of reaching PFS6 is higher in those patients having high XPG protein expression. In fact, 55.6% patients having high XPG expression (expression score 2+ and 3+) reached the PFS6 endpoint meanwhile only 39.9% of the patient with low expression reach PFS6 (expression score 0 and 1+).

Subdivision of the full cohort of patients in two equal subpopulations according to the XPG protein expression produces an increase of the efficacy of ET-743 in the target subpopulation from 13% ( 8/64) to 24% ( 6/25) in objective response (1.85 fold increase) and from 42% ( 27/64) to 60% ( 15/25) in tumor control rate (1.43 fold increase).

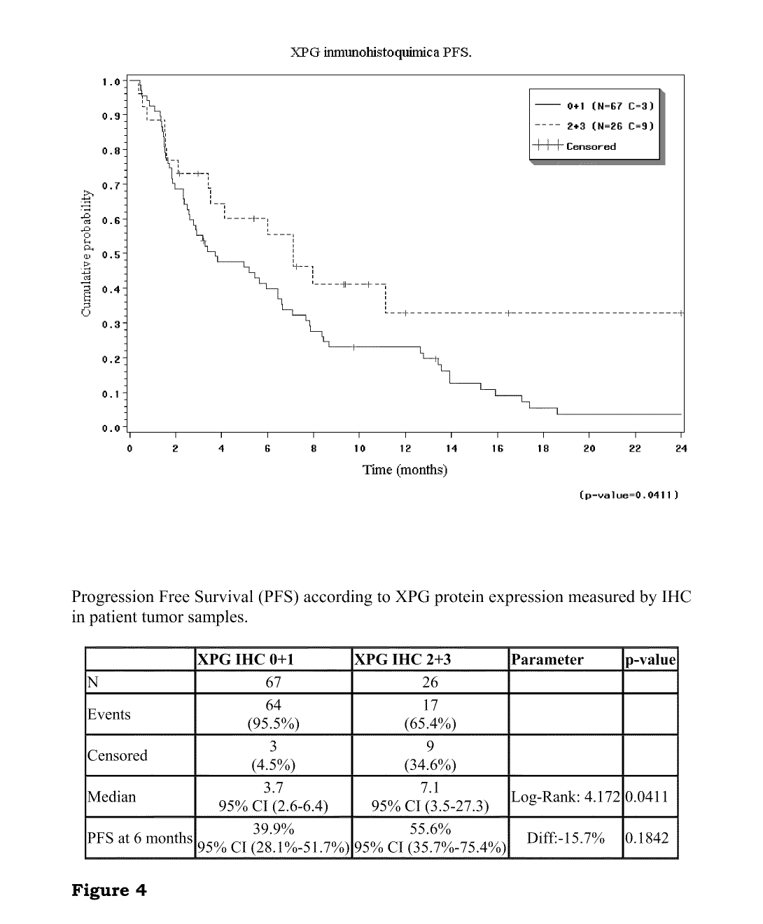

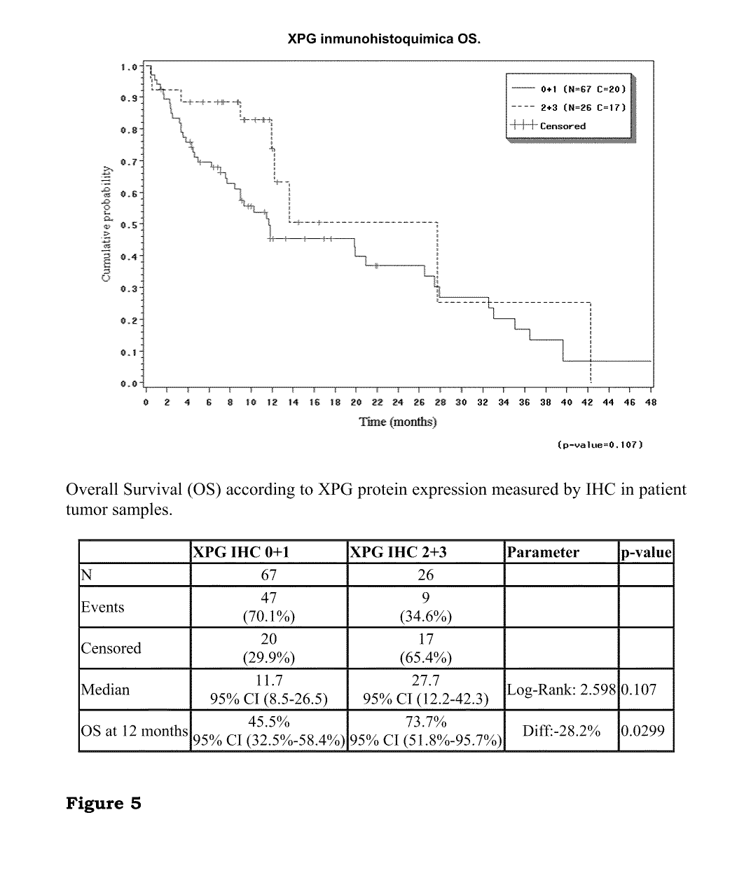

The Kaplan-Meier plots of FIGS. 4 and 5 show a statistically significant difference [p=0.0411] in PFS and a trend [p=0.107] in survival, respectively, on those patients having a high XPG protein expression. The median survival was 27.7 months for high expressers and 11.7 for low expression patients. In addition, the median PFS was 7.1 months for high expression patients and 3.7 months for low expression patients, and the percentage of patients with PFS6 was 55.6% and 39.9%, respectively. The difference in median PFS is statistically significant [p=0.0411], and PFS6 and median survival showed a clear trend to better outcome on high XPG protein expressing patients [p=0.184] and [p=0.107], respectively. Finally, the survival at 12 months was 45.5% for those patients having low XPG protein expression and 73.7% for those patients having high XPG mRNA expression, difference that is statistically significant [p=0.0299].

Correlation of XPG mRNA and BRCA1 mRNA Expression Levels and ET-743 Treatment Outcome.

The amount of XPG mRNA relative to the β-actin (internal control) was determined in 106 samples. This amount was ranging from 0.26 to 8.68, a 33.4-fold difference from the minimum to the maximum value found. The median expression value was 1.55. In these samples the amount of BRCA1 mRNA relative to the β-actin (internal control) was ranging from 0.17 to 17.13, a 100.1-fold difference from the minimum to the maximum value found. The median expression value was 2.36.

The association between the combined expression levels of

XPG and BRCA1 mRNA with the clinical outcome of 106 patients treated with ET-743 is shown in Table 4.

TABLE 4

| | BRCA1 + XPG mRNA | | | | |
|---|---|---|---|---|---|
| | High BRCA1 | High BRCA1 | Low BRCA1 | Low BRCA1 | |
| Parameter | High XPG | Low XPG | High XPG | Low XPG | P-value |
| CR + PR | 5/26 (19%) | 0/20 (0%) | 3/19 (16%) | 6/31 (19%) | 0.1547 |
| CR + PR + MR + SD > 6 | 11/26 (42%) | 4/20 (20%) | 12/19 (63%) | 14/31 (45%) | 0.0575 |
| PFS > 6 Months | 9/26 (35%) | 4/21 (19%) | 10/21 (48%) | 12/27 (61%) | 0.249 |
| rate | | | | | |
| Median PFS | 2.5 m | 2.5 m | 7.3 m | 3.4 m | 0.023 |
| PFS 6 | 38.5% | 22.2% | 55.7% | 35.5% | 0.023 |
| Median Survival | 13.6 m | 6.6 m | 15.4 m | 11.7 m | 0.1587 |

The total patient population was divided in four subpopulations according to the combined expression of BRCA1 and XPG mRNA. Table 4 shows that the subpopulation having low expression levels of BRCA1 (below the median value 2.36) and high expression levels of XPG (above the median value 1.55) has the best outcome after treatment with ET-743. Similarly, the subpopulation having opposite expression of those two genes; that is high expression level of BRCA1 (above the median value 2.36) and low expression level of XPG (below the median value 1.55) has the worst outcome. The two remaining subpopupations have intermediate outcome, comparable to that of the unselected population.

In fact, the low expression BRCA1 and high expression XPG subpopulation (favourable subpopulation) have a better outcome than the opposite one in terms of Median PFS (7.3 vs 2.5 months, p=0.023) and PFS>6 rate (55.7% vs. 22.2%, p=0.023).

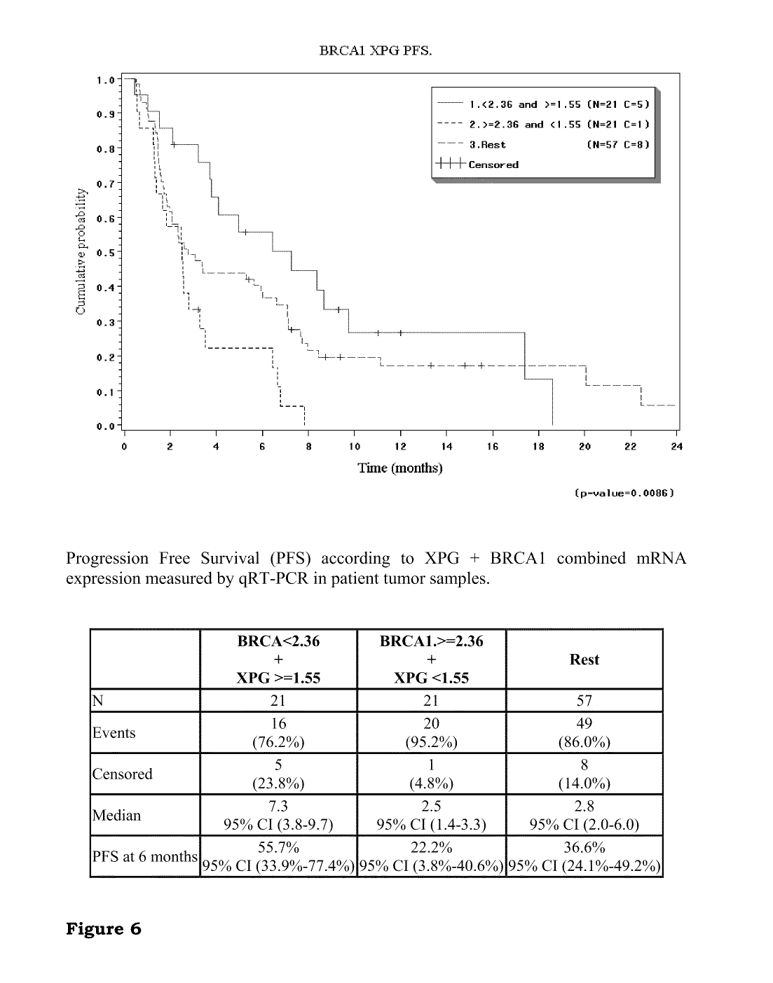

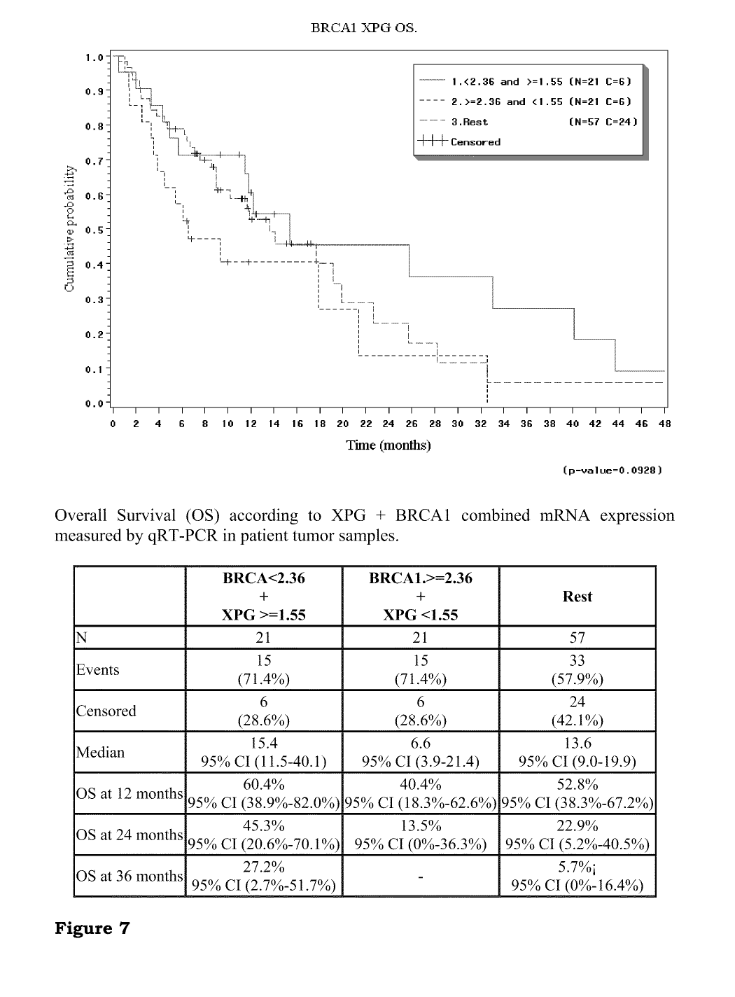

This means that, subdivision of the full cohort of patients in four subpopulations according to the combined expression of BRCA1 and XPG mRNA distinguishes tree different subpopulations according to the outcome after ET-743 treatment (FIGS. 6 and 7). The favourable subpopulation having low expression of BRCA1 and high expression of XPG with median PFS of 7.3 months, PFS6 rate 55.7% and median OS 15.4 months; the unfavourable subpopulation having high expression of BRCA1 and low expression of XPG with median PFS of 2.5 months, PFS6 rate 22.2% and median OS 6.6 months; and a subpopulation with an intermediate prognosis of response to ET-743 (high expression of BRCA1 and high expression of XPG, or low expression of BRCA1 and low expression of XPG) with median PFS of 2.8 months, PFS6 rate 36.6% and median OS 13.6 months.

In conclusion, the expression of XPG, both in terms of mRNA o protein expression, is a biomarker correlated with the clinical outcome of cancer patients treated with ET-743. In fact, subdivision of the full cohort of patients in two subpopulations according to the XPG mRNA expression levels or XPG protein expression levels produces a significant increase of the efficacy of ET-743 in the target subpopulation in terms of objective response, PFS and survival. In addition the prediction of response can be refined if the expression of XPG mRNA is combined with the expression of BRCA1 gene.

### Example 2

### Sample and Clinical Data Collection

In this study, 168 paraffin embedded tumor samples from sarcoma patients before the treatment with any chemotherapy agent have been evaluated.

The majority of patients were treated before with one or several chemotherapy agents and later they followed a treatment with ET-743. The dosage of intravenous infusion (IV) of ET-743 given to the different patients was within the range of 1.650-1.0 mg/m²; the schedules were 24 hours or 3 hour IV infusion with a three week interval between cycles; and the number of cycles ranged from 1 up to 44 cycles in some patients.

The clinical data from the patients was collected in the clinical data collection form and matched with the molecular data (Table 5)

Quantification of XPG mRNA and protein expression levels and quantification of BRCA1 mRNA expression levels was undertaken as described in Example 1.

### Genotyping of Asp1104His SNP at XPG Gene

The genotyping of the single nucleotide polymorphism (SNP) of XPG in paraffin embedded tumor samples was performed by RFLP analysis after PCR amplification of the extracted genomic DNA followed by direct sequencing across the amplified region containing the SNP locus as previously described (Le Morvan et al., Int. J. Cancer: 119, 1732-1735 (2006)). Briefly, PCR products were generated using 10 ng of genomic DNA in 10 ml volume reactions containing 20 mM Tris-HCl, 50 mM KCl, 2.0 mM MgCl2, 0.11 mM each dNTP, 0.3 mM of forward primer TGG ATT TTT GGG GGA GAC CT (SEQ ID NO:8) and of reverse primer CGG GAG CTT CCT TCA CTG AGT (SEQ ID NO:9) and 0.3 U Taq DNA polymerase. The temperature conditions for PCR were set as denaturation at 94° C. for 30 s, annealing 57° C. for 30 s, elongation at 72° C. for 30 s and final extension at 72° C. for 5 min. The amplified fragments were digested with Hsp92II restriction endonuclease. The digested PCR products were resolved on 10% polyacrylamide gels and visualized under UV light after staining with ethidium bromide. The genotype results were regularly confirmed by direct DNA sequencing of the amplified fragments.

Therefore, the wild type (W) genotype or Asp/Asp, was determined by the presence of the nucleotide C in both alleles at the SNP locus rs17655, corresponding to the position 3753 of the mRNA of XPG gene, coding for an Asp at the position 1104 of XPG protein. The variant/mutant (M) genotype was determined by the presence of a G nucleotide in both alleles at the SNP locus and the heterozygous (H) genotype was determined by the presence of C nucleotide in one allele and G in the other allele at the same locus.

### Patient Cohort

A total of 168 paraffin embedded tumor samples providing evaluable results for at least one of the biomarkers (XPGmRNA, XPG protein, XPGAsp1104His or BRCA 1 mRNA) were analysed. Table 5 shows the most relevant clinical and molecular data of the 168 patients (CR: Complete Response; PR: Partial Response; MR: Minor Response; SD: Stable Disease; PD: Progressive Disease; OS: Overall survival; PFS: progression free survival).

TABLE 5
Clinical and molecular data of the 168 patients.

| | | | | | PFS | OS | XPG | XPG | XPG | BRCA1 |
|---|---|---|---|---|---|---|---|---|---|---|
| Patient # | Age | Gender | Tumor Histology | Response | months | months | mRNA | protein | Asp1104His | mRNA |
| 1 | 71 | Male | MFH | SD | 3.2 | 7.7 | — | 0 | H | — |
| 2 | 67 | Male | GIST | PD | 1.3 | 32.6 | 1.31 | | W | 2.84 |
| 3 | 77 | Male | Leiomyosarcoma | SD | 3.4 | 3.8 | 0.56 | 0 | M | 1.44 |
| 4 | 30 | Male | MPNST | PD | 0.5 | 1.1 | 1.27 | 0 | W | 3.95 |
| 5 | 42 | Male | MFH | PD | 1.6 | 2.3 | 0.98 | 1 | W | — |
| 6 | 37 | Male | Liposarcoma | PR | 27.3 | 27.7 | 1.70 | 2 | W | — |
| 7 | 40 | Male | Liposarcoma | PD | 1.7 | 3.3 | 1.09 | 0 | M | 2.71 |
| 8 | 49 | Female | MPNST | PD | 3.7 | 4.3 | 1.96 | 0 | W | 2.12 |
| 9 | 49 | Male | MFH | PD | 1.3 | 1.4 | 0.26 | 0 | W | 3.35 |
| 10 | 50 | Female | MFH | SD | 1.8 | 11.7 | 0.60 | 0 | W | 0.72 |
| 11 | 59 | Male | Liposarcoma | PD | 0.6 | 0.6 | — | 2 | W | 3.37 |
| 12 | 37 | Female | MFH | PD | 1.7 | 1.7 | 0.91 | 0 | W | 0.74 |
| 13 | 36 | Female | Leiomyosarcoma | PD | 1.4 | 3.3 | 0.76 | 0 | W | — |
| 14 | 28 | Female | Synovial Sarcoma | PD | 2.5 | 2.5 | 1.28 | 0 | W | 3.11 |
| 15 | 55 | Female | Leiomyosarcoma | PD | 1.4 | 4.5 | 0.47 | 0 | W | 1.40 |
| 16 | 34 | Female | Synovial Sarcoma | PD | 2.4 | 3.2 | 2.47 | 0 | H | 4.02 |
| 17 | 54 | Female | Liposarcoma | PD | 3.3 | 3.5 | 0.70 | 1 | H | 3.55 |
| 18 | 45 | Female | Synovial Sarcoma | SD | 13.6 | 26.5 | — | 0 | W | 0.81 |
| 19 | 32 | Male | Synovial Sarcoma | SD | 17.4 | 20.9 | — | 0 | H | — |
| 20 | 64 | Female | Synovial Sarcoma | SD | 6.4 | 11.5 | 3.91 | 0 | W | 2.19 |
| 21 | 54 | Male | Leiomyosarcoma | PD | 1.1 | 11.8 | — | 0 | | — |
| 22 | 52 | Male | Osteosarcoma | PD | 1.5 | 4.3 | — | 0 | | — |
| 23 | 46 | Female | Other | PD | 2.5 | 6.1 | 1.51 | | | 2.53 |
| 24 | 33 | Male | Osteosarcoma | PD | 2.8 | 6.6 | 0.53 | | | 2.75 |
| 25 | 57 | Female | Myxoid | PR | 22.4 | 28.2 | 1.96 | | | 4.33 |
| | | | Liposarcoma | | | | | | | |
| 26 | 29 | Female | Other | PD | 1.3 | 11.9 | 2.01 | | | 4.81 |
| 27 | 44 | Female | Synovial Sarcoma | PD | 0.7 | 58.4 | 0.66 | | | — |
| 28 | 56 | Female | Leiomyosarcoma | PD | 1.3 | 21.4 | 0.31 | | | 4.55 |
| 29 | 22 | Male | Osteosarcoma | PD | 1.5 | 2.9 | 1.40 | | | — |
| 30 | 54 | Female | Liposarcoma | PD | 0.6 | 1.4 | 1.09 | | | 8.96 |
| 31 | 36 | Female | Ewing Sarcoma | PD | 2.6 | 8.2 | — | | W | — |
| 32 | 18 | Female | Synovial Sarcoma | SD | 6.8 | 17.9 | 1.54 | | | 9.22 |
| 33 | 54 | Male | Liposarcoma | PD | 1.1 | 10.2 | 1.59 | | | 10.12 |
| 34 | 20 | Male | Other | SD | 3.2 | 40.1 | 1.66 | | | 1.38 |
| 35 | 64 | Male | Other | SD | 7.7 | 25.7 | 0.85 | | | 1.54 |
| 36 | 35 | Female | Ewing Sarcoma | PD | 1.0 | 17.7 | 1.49 | | | 2.19 |
| 37 | 43 | Male | Other | PD | 1.0 | 22.7 | 2.21 | | | 2.37 |
| 38 | 26 | Female | Synovial Sarcoma | PR | 3.1 | 19.1 | 2.71 | | | 3.68 |
| 39 | 38 | Male | Other | SD | 4.1 | 15.4 | 3.17 | | M | 2.03 |
| 40 | 34 | Female | Synovial Sarcoma | PD | 2.1 | 4.7 | 2.33 | | | 4.14 |
| 41 | 30 | Male | Other | SD | 7.8 | 37.9 | 2.85 | | | — |
| 42 | 38 | Female | Uterine | PD | 1.5 | 6.7 | 0.75 | | | 0.92 |
| | | | Leiomyosarcoma | | | | | | | |
| 43 | 40 | Female | MPNST | PD | 2.1 | 2.4 | 1.35 | | | 1.13 |
| 44 | 19 | Male | Ewing Sarcoma | PD | 0.7 | 4.5 | 0.61 | | | 3.92 |
| 45 | 28 | Male | Other | PD | 1.4 | 5.4 | 0.51 | | | 4.53 |
| 46 | 46 | Male | Leiomyosarcoma | PD | 0.6 | 0.8 | — | | W | — |
| 47 | 56 | Male | Osteosarcoma | PD | 2.4 | 10.4 | — | | W | — |
| 48 | 28 | Female | Other | SD | 7.5 | 21.7 | 1.98 | | | — |
| 49 | 38 | Male | Other | PD | 1.0 | 1.8 | — | | H | — |
| 50 | 35 | Male | Other | PD | 0.7 | 3.2 | 2.43 | | | 2.81 |
| 51 | 41 | Female | Uterine | PR | 17.4 | 43.7 | 3.02 | | | 0.58 |
| | | | Leiomyosarcoma | | | | | | | |
| 52 | 21 | Male | Osteosarcoma | PD | 1.5 | 6.4 | 2.99 | | | 5.66 |
| 53 | 39 | Female | Other | PD | 0.6 | 0.7 | — | | M | — |
| 54 | 18 | Female | Ewing Sarcoma | PD | 0.9 | 6.4 | — | | H | — |
| 55 | 29 | Female | Other | PD | 1.6 | 6.9 | — | | H | 2.76 |
| 56 | 42 | Female | Osteosarcoma | SD | 7.1 | 14.1 | 3.65 | | | 8.23 |
| 57 | 66 | Female | Other | MR | 3.8 | 5.7 | 3.86 | | | 2.12 |
| 58 | 60 | Female | Other | MR | 9.7 | 25.8 | 2.26 | | | 2.32 |
| 59 | 37 | Female | Other | PD | 0.7 | 1.2 | 1.76 | | | 5.13 |
| 60 | 38 | Female | Other | MR | 2.5 | 16.4 | 0.80 | | | — |
| 61 | 59 | Male | Myxoid | PR | 16.5 | 16.5 | — | 2 | | — |
| | | | Liposarcoma | | | | | | | |
| 62 | 65 | Male | Myxoid | PR | 3.0 | 14.5 | — | 2 | W | — |
| | | | Liposarcoma | | | | | | | |
| 63 | 35 | Female | Leiomyosarcoma | PR | 8.7 | 11.8 | 1.69 | 0 | | 0.64 |
| 64 | 52 | Male | Myxoid | PR | 15.5 | 15.5 | 3.79 | | W | 5.92 |
| | | | Liposarcoma | | | | | | | |
| 65 | 52 | Male | Myxoid | PR | 15.5 | 15.5 | 4.41 | | W | 3.69 |
| | | | Liposarcoma | | | | | | | |
| 66 | 56 | Male | Myxoid | MR | 6.6 | 15.1 | 0.79 | 0 | H | 1.59 |
| | | | Liposarcoma | | | | | | | |
| 67 | 64 | Female | Uterine | PR | 5.9 | 7.1 | 1.04 | 0 | | 0.83 |
| | | | Leiomyosarcoma | | | | | | | |
| 68 | 56 | Male | Other | PD | 0.5 | 0.5 | 1.59 | 0 | | 3.13 |
| 69 | 20 | Female | sacro-iliacal right | PD | 1.5 | 1.5 | — | 0 | | — |
| 70 | 70 | Female | Leiomyosarcoma | PD | 0.5 | 0.5 | 1.87 | 0 | | 0.92 |
| 71 | 70 | Female | Leiomyosarcoma | SD | 8.7 | 8.7 | 0.41 | | | 2.35 |
| 72 | 54 | Male | Liposarcoma | SD | 3.2 | 11.8 | 1.14 | 0 | | 2.73 |
| 73 | 51 | Female | Myxoid | PR | 7.1 | 7.1 | 1.15 | 1 | | 0.80 |
| | | | Liposarcoma | | | | | | | |
| 74 | 53 | Female | Uterine | PD | 2.3 | 11.3 | 0.43 | 0 | | 0.38 |
| | | | Leiomyosarcoma | | | | | | | |
| 75 | 74 | Female | Myxoid | MR | 5.3 | 12.4 | 2.45 | | | 0.94 |
| | | | Liposarcoma | | | | | | | |
| 76 | 54 | Male | Other | SD | 2.2 | 12.5 | 1.67 | 2 | | 0.58 |
| 77 | 60 | Female | Leiomyosarcoma | PD | 2.3 | 12.1 | 1.11 | 1 | | 1.55 |
| 78 | 40 | Male | Liposarcoma | MR | 14.8 | 17.2 | 1.28 | | | 2.29 |
| 79 | 25 | Male | Other | PR | 7.3 | 14.0 | 2.23 | | | 2.34 |
| 80 | 42 | Male | Other | PD | 1.7 | 8.0 | 1.08 | | | 0.37 |
| 81 | 56 | Male | Myxoid | MR | 6.1 | 11.6 | 1.07 | | | — |
| | | | Liposarcoma | | | | | | | |
| 82 | — | | Osteosarcoma | NA/NK/NE | — | — | — | 0 | | 3.40 |
| 83 | — | | Leiomyosarcoma | NA/NK/NE | — | — | — | 0 | | 3.54 |
| 84 | — | | Leiomyosarcoma | NA/NK/NE | — | — | — | 0 | | 4.86 |
| 85 | — | Male | Osteosarcoma | NA/NK/NE | — | — | 0.99 | | | 2.38 |
| 86 | 69 | Male | Myxoid | PR | 11.1 | 11.1 | 1.47 | 2 | H | 2.19 |
| | | | Liposarcoma | | | | | | | |
| 87 | 53 | Male | Myxoid | PD | 1.5 | 9.4 | 3.08 | | | 3.69 |
| | | | Liposarcoma | | | | | | | |
| 88 | 58 | Male | Myxoid | PD | 3.4 | 13.6 | 2.17 | 2 | H | 2.73 |
| | | | Liposarcoma | | | | | | | |
| 89 | 59 | Female | Other | PD | 1.5 | 2.3 | 1.05 | 0 | M | 1.74 |
| 90 | 57 | Female | Leiomyosarcoma | PR | 9.7 | 9.7 | — | 0 | W | 0.50 |
| 91 | 70 | Male | Liposarcoma | SD | 10.4 | 10.4 | 4.51 | 2 | W | — |
| 92 | 43 | Female | Myxoid | SD | 8.0 | 8.8 | 1.27 | 2 | W | 2.20 |
| | | | Liposarcoma | | | | | | | |
| 93 | — | | | PD | 1.5 | 1.6 | 2.92 | 1 | H | 3.68 |
| 94 | 45 | Female | Uterine | PD | 2.5 | 3.9 | 0.89 | | H | 3.66 |
| | | | Leiomyosarcoma | | | | | | | |
| 95 | 47 | Female | Uterine | SD | 15.9 | 36.5 | — | 0 | | — |
| | | | Leiomyosarcoma | | | | | | | |
| 96 | 61 | Male | MFH | SD | 42.0 | 42.3 | — | 2 | | — |
| 97 | 18 | Female | Other | PR | 20.1 | 52.7 | 0.71 | | H | 0.65 |
| 98 | 42 | Male | Other | PD | 5.4 | 19.8 | — | 1 | | — |
| 99 | — | Female | Liposarcoma | NA/NK/NE | — | — | 2.53 | | | 9.34 |
| 100 | 50 | Female | | PR | 13.4 | 27.4 | — | 1 | | 17.13 |
| 101 | 45 | Male | | SD | 7.9 | 35.1 | — | 0 | | 1.98 |
| 102 | 35 | Female | | MR | 29.0 | 64.1 | — | 0 | | — |
| 103 | — | | | NA/NK/NE | — | — | — | 0 | | — |
| 104 | 60 | Male | Myxoid | SD | 9.3 | 9.3 | 3.10 | 2 | | 1.62 |
| | | | Liposarcoma | | | | | | | |
| 105 | 55 | Female | Leiomyosarcoma | SD | 7.8 | 10.0 | 0.68 | 0 | | 2.62 |
| 106 | 72 | Female | Leiomyosarcoma | SD | 6.7 | 10.0 | 1.30 | 0 | W | 2.56 |
| 107 | 62 | Male | Liposarcoma | SD | 11.0 | 11.0 | 5.68 | | W | 1.25 |
| 108 | 55 | Male | Liposarcoma | PD | 2.0 | 2.4 | 1.71 | 0 | H | 3.42 |
| 109 | 47 | Male | Myxoid | PR | 15.3 | 22.0 | — | 0 | H | — |
| | | | Liposarcoma | | | | | | | |
| 110 | 74 | Male | Other | SD | 7.3 | 7.3 | 7.54 | 2 | W | 2.96 |
| 111 | 53 | Female | GIST | PD | 2.6 | 6.9 | 1.19 | 0 | W | 4.85 |
| 112 | 61 | Female | Uterine | PD | 3.5 | 6.8 | 0.91 | 2 | H | 2.85 |
| | | | Leiomyosarcoma | | | | | | | |
| 113 | 69 | Male | Myxoid | PR | 7.1 | 7.4 | 8.15 | 2 | H | 3.72 |
| | | | Liposarcoma | | | | | | | |
| 114 | 54 | Female | Uterine | NA/NK/NE | — | — | 0.71 | 1 | W | 3.35 |
| | | | Leiomyosarcoma | | | | | | | |
| 115 | 70 | Male | Leiomyosarcoma | SD | 8.4 | 33.0 | 4.29 | 0 | | 2.16 |
| 116 | 48 | Male | Myxoid | SD | 2.1 | 3.3 | 1.62 | 2 | H | 0.77 |
| | | | Liposarcoma | | | | | | | |
| 117 | 62 | Female | Synovial Sarcoma | SD | 4.1 | 12.0 | 4.30 | 2 | M | — |
| 118 | 70 | Male | Rabdomiosarcoma | NA/NK/NE | 0.4 | 0.4 | — | 2 | M | — |
| 119 | 47 | Male | Liposarcoma | PD | 1.6 | 9.0 | 1.40 | 3 | M | 0.62 |
| 120 | 65 | Male | GIST | PD | 1.4 | 19.9 | 1.86 | 0 | W | 2.82 |
| 121 | 49 | Female | Leiomyosarcoma | SD | 7.7 | 9.0 | 1.36 | 0 | H | 1.30 |
| 122 | 54 | Female | Leiomyosarcoma | SD | 3.8 | 8.5 | — | 0 | | — |
| 123 | 23 | Female | Synovial Sarcoma | SD | 18.6 | 53.5 | 2.93 | 0 | W | 1.50 |
| 124 | 39 | Male | Other | NA/NK/NE | 0.9 | 0.9 | — | 0 | | — |
| 125 | 25 | Female | Other | PR | 17.1 | 27.9 | 2.53 | 0 | W | — |
| 126 | 63 | Male | MFH | SD | 29.7 | 32.6 | 1.32 | 0 | W | 0.92 |
| 127 | 33 | Male | Other | PD | 2.9 | 4.6 | 1.55 | 0 | H | — |
| 128 | 35 | Male | Other | SD | 13.9 | 39.6 | — | 0 | W | — |
| 129 | 65 | Female | Leiomyosarcoma | PD | 1.9 | 7.6 | 0.28 | 0 | W | 0.84 |
| 130 | 33 | Male | GIST | NA/NK/NE | 5.0 | 5.0 | 2.22 | 0 | H | 1.26 |
| 131 | 70 | Female | Leiomyosarcoma | NA/NK/NE | 1.8 | 9.3 | 1.15 | 1 | M | 4.09 |
| 132 | 43 | Female | Leiomyosarcoma | SD | 5.6 | 16.9 | 0.53 | 0 | M | 0.17 |
| 133 | 59 | Female | Liposarcoma | SD | 12.0 | 12.0 | 2.84 | 2 | H | 1.60 |
| 134 | 47 | Female | Leiomyosarcoma | PD | 1.6 | 11.8 | 0.27 | 2 | M | 1.43 |
| 135 | 62 | Female | Leiomyosarcoma | PD | 2.8 | 6.2 | 0.37 | 0 | H | 0.29 |
| 136 | 57 | Female | Liposarcoma | SD | 5.2 | 10.3 | — | 0 | H | — |
| 137 | 52 | Male | Liposarcoma | PR | 6.0 | 8.7 | 1.29 | 3 | W | 0.31 |
| 138 | 73 | Male | Liposarcoma | SD | 13.3 | 13.3 | 3.31 | 0 | W | 2.63 |
| 139 | — | | Leiomyosarcoma | NA/NK/NE | — | — | 0.93 | | | — |
| 140 | — | | Leiomyosarcoma | NA/NK/NE | — | — | 0.53 | | | — |
| 141 | 9 | Male | Ewing Sarcoma | NA/NK/NE | — | — | 1.84 | | | 3.20 |
| 142 | — | | Myxoid | NA/NK/NE | — | — | — | | H | 3.11 |
| | | | Liposarcoma | | | | | | | |
| 143 | — | | Ewing Sarcoma | NA/NK/NE | 1.0 | 2.0 | 5.46 | | H | 1.71 |
| 144 | — | | Liposarcoma | NA/NK/NE | — | — | 2.47 | | W | 2.66 |
| 145 | 74 | Male | Liposarcoma | PD | 0.8 | 4.2 | — | 0 | | — |
| 146 | 74 | Male | Liposarcoma | PD | 0.8 | 4.2 | — | 2 | | — |
| 147 | 72 | Male | Liposarcoma | CR | 12.8 | 17.6 | — | 0 | | — |
| 148 | 47 | Male | | PD | 0.3 | 0.7 | 5.54 | | | — |
| 149 | 70 | Male | | SD | 11.9 | 19.5 | 6.15 | | | — |
| 150 | 56 | Male | | PR | 47.4 | 65.9 | 1.55 | | | — |
| 151 | 54 | Male | Leiomyosarcoma | SD | 8.4 | 9.0 | 0.53 | 0 | W | 0.62 |
| 152 | 57 | Female | Leiomyosarcoma | PD | 0.7 | 8.6 | 0.29 | | W | 0.65 |
| 153 | 56 | Female | Uterine | MR | 6.4 | 6.4 | 0.94 | 0 | H | 4.58 |
| | | | Leiomyosarcoma | | | | | | | |
| 154 | 54 | Female | Uterine | SD | 7.6 | 11.9 | 0.30 | | W | — |
| | | | Leiomyosarcoma | | | | | | | |
| 155 | 42 | Female | Uterine | PD | 2.9 | 5.2 | — | 0 | W | — |
| | | | Leiomyosarcoma | | | | | | | |
| 156 | 36 | Female | Myxoid | SD | 7.1 | 11.2 | 4.91 | 3 | W | 2.36 |
| | | | Liposarcoma | | | | | | | |
| 157 | 34 | Male | Myxoid | SD | 12.6 | 21.8 | 3.03 | 1 | W | — |
| | | | Liposarcoma | | | | | | | |
| 158 | 48 | Female | Uterine | PD | 1.4 | 5.8 | 0.65 | | W | — |
| | | | Leiomyosarcoma | | | | | | | |
| 159 | 52 | Male | Myxoid | PD | 1.5 | 12.2 | 2.40 | 3 | M | 0.35 |
| | | | Liposarcoma | | | | | | | |
| 160 | 59 | Female | Uterine | PR | 5.3 | 11.5 | 0.88 | | | 1.19 |
| | | | Leiomyosarcoma | | | | | | | |
| 161 | 28 | Female | Uterine | SD | 2.6 | 9.1 | 1.56 | 0 | W | 3.11 |
| | | | Leiomyosarcoma | | | | | | | |
| 162 | 70 | Male | Myxoid | SD | 9.4 | 9.4 | 8.68 | 3 | W | 2.84 |
| | | | Liposarcoma | | | | | | | |
| 163 | 35 | Female | Chondrosarcoma | NA/NK/NE | — | — | 3.94 | 2 | | 1.67 |
| 164 | 35 | Male | Other | SD | 13.9 | 39.6 | 1.56 | 0 | | — |
| 165 | 38 | Male | Myxoid | MR | 5.4 | 5.4 | 8.26 | 3 | W | 7.00 |
| | | | Liposarcoma | | | | | | | |
| 166 | 15 | Male | Ewing Sarcoma | NA/NK/NE | — | — | 3.45 | | | 6.75 |
| 167 | — | | | NA/NK/NE | — | — | 7.23 | | M | — |
| 168 | — | Male | | NA/NK/NE | — | — | 1.43 | | | — |

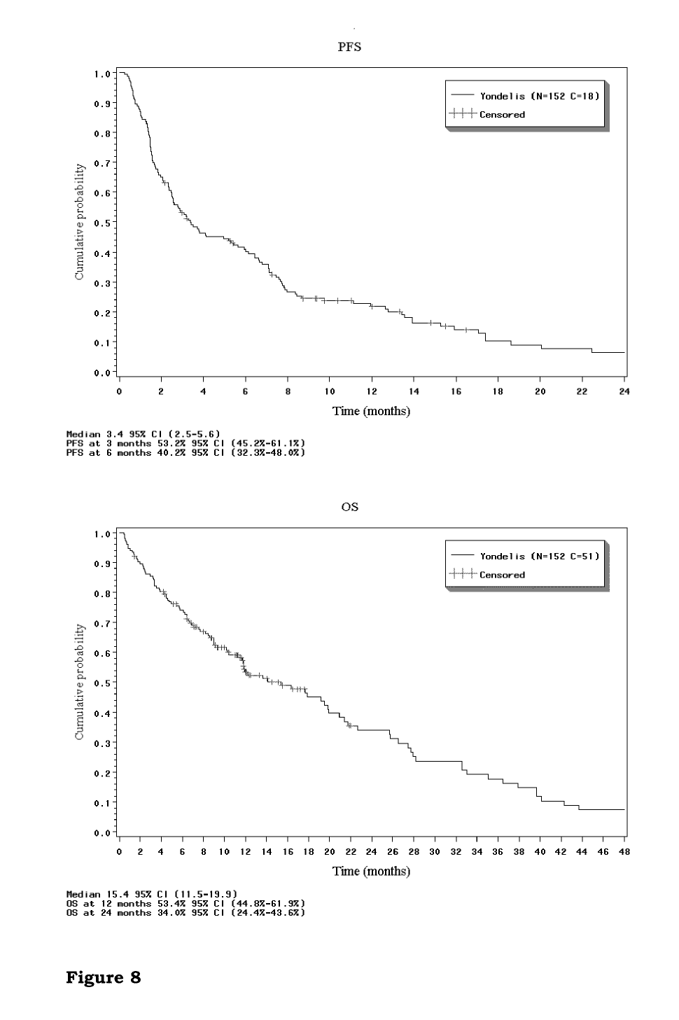

After treatment with ET-743, the overall response rate (RR) in 147 evaluable patients (21 patients (12.5%) with unknown or non evaluable response) was 15.6% when considering Complete+Partial Responses ((1 CR+22 PR)/ 147 evaluable patients). In addition, 6.8% patients had Minor Responses (MR) (10 MR/147) and 31.29% (46/147 patients) had Stable Disease (SD). Tumor Control Rate ((CR+PR+MR+SD≧6 mo) was achieved in 44.9% of the patients (66/147) and 58 patients (39.5%) achieved progression free survival ≧6 months (PFS6). The median duration of the response (CR+PR+MR) was 7.83 months (range 47.4 to 1.83 months) and 33 out of 46 SD reached the PFS6. Median survival was 9.6 months (0.4-65.9 months), although 51 patients are still censored. According to the Kaplan-Meier plots (FIG. 8), the median progression free survival was 3.4 months and the PFS6 rate is 40.2% and the median survival is 15.4 months, with 5.34% and 34.0% of surviving patients at 1 and two years after treatment.

### Correlation of XPG SNP Asp1104His Genotype and ET-743 Treatment Outcome.

The genotype of the XPG SNP Asp1104His was determined in 87 samples of paraffin embedded tumor tissue from sarcoma patients treated with ET-743, as described in table 5.

The genotypes were designed as wild type (W) or Asp/Asp by the presence of the nucleotide C in both alleles at the SNP locus rs17655, corresponding to the position 3753 of the mRNA of XPG gene (SEQ ID NO:1), coding for a Asp at the position 1104 of XPG protein. The variant/mutant (M) genotype was determined by the presence of a G nucleotide in both alleles at the SNP locus. The heterozygous (H) genotype was determined as the presence of C nucleotide in one allele and G in the other allele at the same locus. The association between the three genotypes W, M and H with the clinical outcome of the patients treated with ET-743, is shown in Table 6.

TABLE 6

| | XPG genotype Asp1104His | | | |
|---|---|---|---|---|
| Parameter | W | H | M | p-Value |
| CR + PR | 7 (15.6%) | 4 (16.7%) | 0 | 0.4702 |
| CR + PR + MR + SD ≧ 6 | 24 (53.3%) | 9 (37.5%) | 0 | 0.0046 |
| PFS ≧ 6 Months | 22 (48.9%) | 9 (34.6%) | 0 | 0.0037 |
| Median PFS (KM) | 6.4 | 3.4 | 1.6 | 0.0010 |
| PFS6 (KM) | 50.8% | 34.6% | 0% | 0.0010 |
| Median Survival | 19.9 | 6.9 | 9.1 | 0.0743 |
| (KM) | | | | |

KM: according to the Kaplan-Meier plots

Table 6 shows that the highest rates for Objective Response and Tumor Control (16% and 53%) are found in patients having W genotype of XPG SNP Asp1104His, compared with 17% and 38% in the H genotype. The patients having M genotype did not respond to ET-743 treatment.

Similarly, the probability of reaching PFS6 is highest in those patients having W genotype. In particular, 49% of patients having W genotype reached the PFS6 endpoint meanwhile only 35% of the patient with H genotype and none of the M genotype reached FPS6.

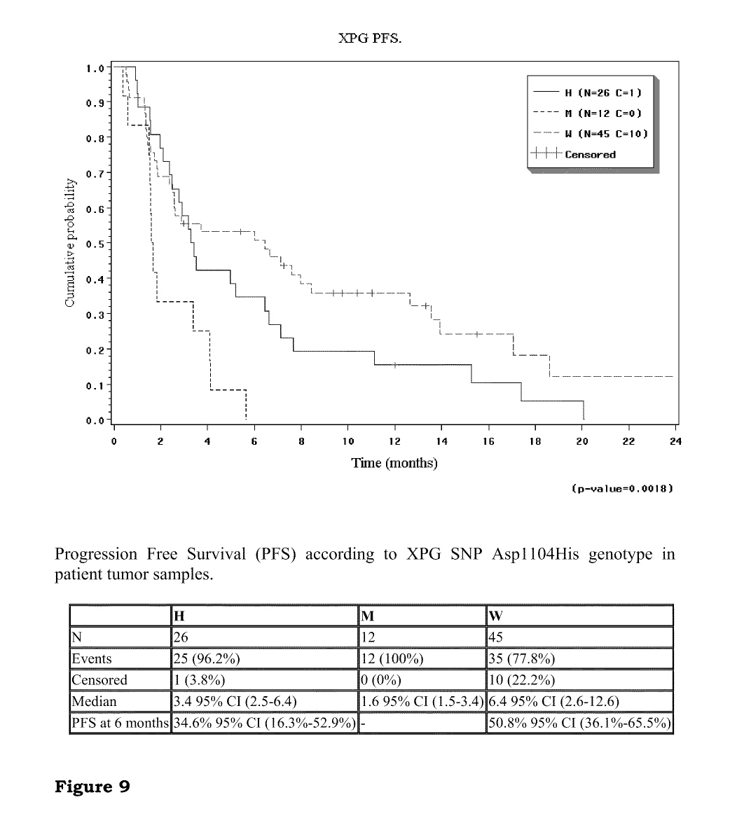

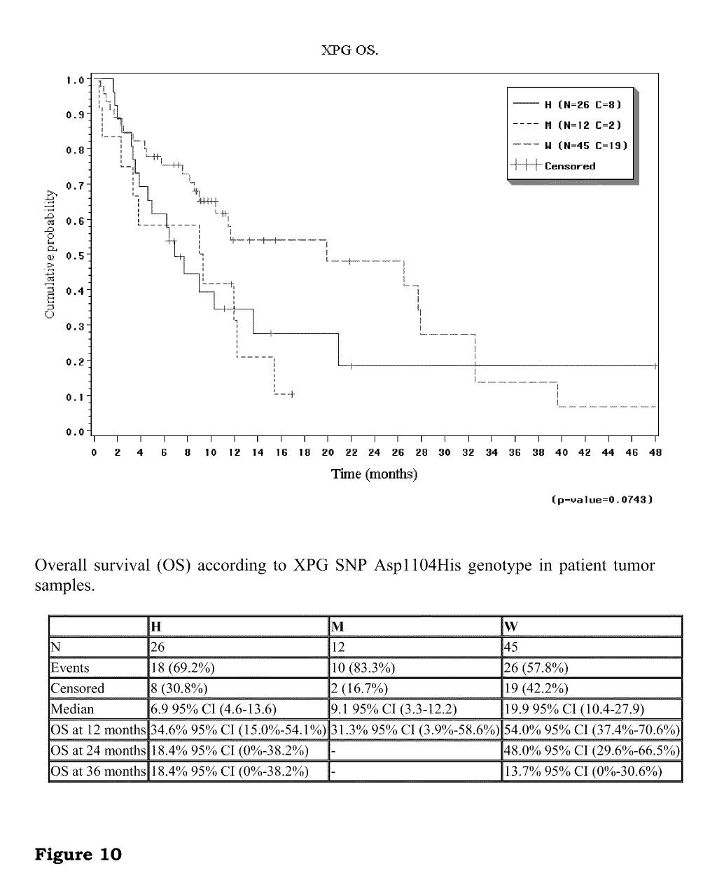

The Kaplan-Meier plots of FIGS. 9 and 10 show a statistically significant difference [p=0.0010] in PFS and a trend [p=0.0743] in survival, respectively, on those patients having patients having W genotype, compared to H and M genotypes. The median survival was 19.9 months for patients having W genotype and 6.9 and 9.1 for patients having H and M genotypes. In addition, the median PFS was 6.4 months for patients having W genotype and 3.4 and 1.6 for patients having H and M genotypes with PFS6 rates of 50.8%, 34.6% and 0%, respectively for the W, H and M genotypes. Finally, the survival at 12 months was 54% for those patients having W genotype and 34.6% and 31.3% for those patients having H and M genotypes.

This means that the subdivision of the full cohort of patients in 3 subpopulations according to the genotype for the Asp1104His SNP of XPG gene distinguishes tree different subpopulations according to the outcome after ET-743 treatment. The subpopulation with the most favourable clinical outcome, defined by the presence of W genotype; the subpopulation showing no benefit from ET-743 treatment, defined by the M genotype and a third subpopulation with intermediate outcome, corresponding to the heterozygous patients for the Asp1104His SNP.

In the subsequent biomarker association studies including the Asp1104His genotype, the H+M genotypes were grouped to minimize the number of subgroups for a more clear analysis, having also in mind that the objective is identifying the subpopulation obtaining the highest benefit from ET-743 treatment.

Correlation of the Combined XPG mRNA Levels and the XPG SNP Asp1104His Genotype and Response to ET-743 Treatment

The amount of XPG mRNA relative to the β-actin (internal control) was determined in 106 samples, as described in Table 5. This amount was ranging from 0.26 to 8.68, a 33.4-fold difference from the minimum to the maximum value found. The median expression value was 1.55.

The association between the combined expression levels of XPG mRNA and the genotype of XPG SNP Asp1104His with the clinical outcome of 65 patients treated with ET-743 is shown in Table 7.

TABLE 7

| | Asp/His + His/His XPG | | | | |
|---|---|---|---|---|---|
| | (H + M) | | Asp/Asp XPG (W) | | |
| | XPG < 1.55 | XPG >= 1.55 | XPG < 1.55 | XPG >= 1.55 | p-value |
| N | 16 | 13 | 19 | 17 | |
| Median PFS | 3.3 | 2.9 | 1.8 | 17.1 | 0.0011 |
| | 95% CI (1.8-6.4) | 95% CI (2.0-4.1) | 95% CI (1.4-6.0) | 95% CI (7.1-27.3) | |
| PFS at 6 | 31.3% | 15.4% | 26.3% | 82.4% | 0.0011 |
| months | 95% CI (8.5%-54.0%) | 95% CI (0%-35.0%) | 95% CI (6.5%-46.1%) | 95% CI (64.2%-100%) | |
| Median OS | 9.0 | 5.0 | 8.6 | 27.7 | 0.0053 |
| | 95% CI (3.9-) | 95% CI (3.2-13.6) | 95% CI (3.3-32.6) | 95% CI (19.9-53.5) | |
| OS at 12 | 39.1% | 36.9% | 28.9% | 83.7% | 0.0053 |
| months | 95% CI (13.4%-64.7%) | 95% CI (9.9%-64.0%) | 95% CI (4.9%-53.0%) | 95% CI (61.9%-100%) | |

The total patient population was divided in four subpopulations according to the combined expression of XPG and the genotype of XPG SNP Asp1104His. In order to make easier the interpretation of the results with regards to the Asp1104His SNP genotype, genotypes H and M (carrying the mutation in at least one of the alleles) were grouped together and compared with the W genotype. Table 7 shows that the subpopulation having high expression levels of XPG mRNA (above the median value 1.55) together with a W genotype has the best outcome after treatment with ET-743.

In particular, the subpopulation with high expression of XPG mRNA and a W genotype (favourable subpopulation) has a better outcome than the other subpopulations (M+H genotype and low XPG mRNA expression, M+H genotype and high XPG mRNA expression and W genotype and low XPG mRNA expression) in terms of Median PFS (17.1 vs 3.3, 2.9 and 1.8 months respectively, p=0.0011) and of PFS>6 rate (82.4% vs. 31.3, 15.4 and 26.3 months respectively, p=0.0011). The median OS was also statistically significant better in the subpopulation with high expression XPG mRNA and W genotype, compared to the other 3 subpopulations which showed similar values for median OS (27.7 months for the favourable subpopulation compared to 9.0, 5.0 and 8.6 months respectively, p=0.0053). Similarly, the rate of survival after 1 year (83.7% vs 39.1%, 36.9% and 28.9%) and 2 years (66.9% vs 39.1%, 0% and 28.9%) was significantly (p=0.0053) favourable for the high expression of XPG mRNA and the W genotype.

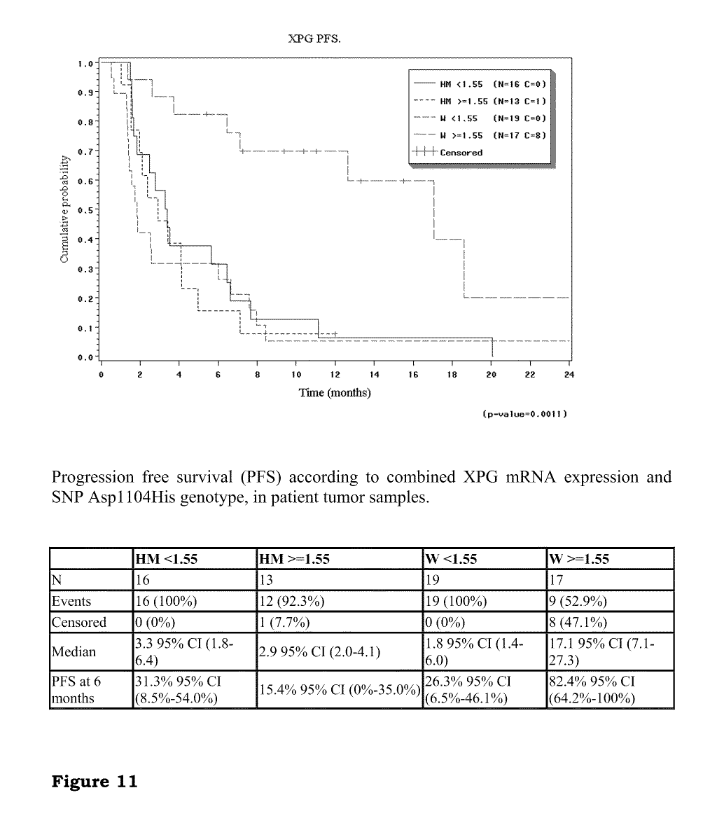

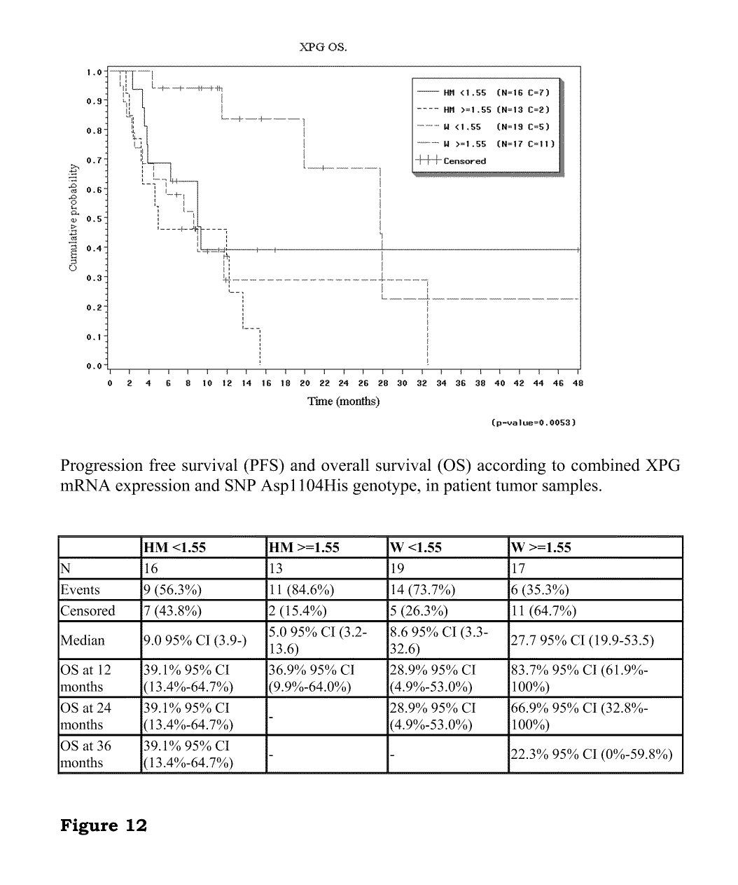

This means that, the subdivision of the full cohort of patients in four groups according to the combined expression of XPG mRNA and the genotype for the Asp1104His SNP distinguishes two different subpopulations according to the outcome after ET-743 treatment, i.e. better and worse responders (FIGS. 11 and 12). The most favourable subpopulation having high expression of XPG mRNA and the W genotype with clearly a better clinical outcome compared to a less favourable subpopulation comprising the patients belonging to the other three groups defined as the combined M+H genotype and low XPG mRNA expression, M+H genotype and high XPG mRNA expression and W genotype and low XPG mRNA expression, respectively.

### Correlation of XPG Protein Expression Levels and Genotype at the SNP Asp1104His of XPG and ET-743 Treatment Outcome.

The amount of XPG protein was determined in 92 samples of paraffin embedded tumor tissue from sarcoma patients treated with ET-743. The score of expression was determined as 0 (no expression of XPG protein), 1+(low expression), 2+(moderate expression) and 3+(high expression). The number of samples in each expression scoring group was 58 (60.0%), 9 (9.5%), 20 (21.7%) and 5 (5.4%), respectively.

In order to test the association between the expression levels of XPG protein with the clinical outcome of the patients treated with ET-743, the patients were grouped in two subpopulations: the low expressers those having low or no expression of XPG proteins (Scores 0 and 1+: 67 patients) and the high expressers having intermediate and high expression (scores 2+and 3+: 25 patients).

The genotype of the XPG SNP Asp1104His was determined in 87 of the paraffin embedded tumor tissue samples from sarcoma patients previous to the treatment with ET-743. Both the correlation of XPG protein expression and the XPG SNP Asp1104His genotype was determined in 66 tissue samples, as described in Table 5.

The total patient population was divided in four subpopulations according to the combined expression of XPG protein and the genotype of XPG SNP Asp1104His. For an easier interpretation, the XPG protein expression was divided in two groups; the low expressers those having low or no expression of XPG proteins (Scores 0 and 1+) and the high expressers having intermediate and high expression (scores 2+and 3+: 25 patients). Similarly, with regards to the XPG SNP Asp1104His, the genotypes H and M were grouped together and compared with the W genotype.

The association between the combined XPG protein expression levels and XPG SNP Asp1104His genotype with the clinical outcome of patients treated with ET-743 is shown in Table 8.

TABLE 8

| | Asp/His + His/His XPG | | | | |
|---|---|---|---|---|---|
| | (H + M) | | Asp/Asp XPG (W) | | |
| | XPG 0 + 1 | XPG 2 + 3 | XPG 0 + 1 | XPG 2 + 3 | p-value |
| N | 19 | 11 | 25 | 10 | |
| Median PFS | 3.3 | 3.4 | 2.9 | 8.0 | 0.0682 |
| | 95% CI (2.4-5.6) | 95% CI (1.6-7.1) | 95% CI (1.8-12.6) | 95% CI (6.0-27.3) | 0.0682 |
| PFS at 6 | 26.3% | 27.3% | 44.0% | 77.1% | 0.0682 |
| months | 95% CI | 95% CI | 95% CI | 95% CI | |
| | (6.5%-46.1%) | (1.0%-53.6%) | (24.5%-63.5%) | (48.9%-100%) | |
| Median OS | 6.2 | 12.2 | 11.7 | 27.7 | 0.0697 |
| | 95% CI (3.5-10.3) | 95% CI (9.0-13.6) | 95% CI (4.5-27.9) | 95% CI (—) | |
| OS at 12 | 23.7% | 52.6% | 47.1% | 90.0% | 0.0697 |
| months | 95% CI | 95% CI | 95% CI | 95% CI | |
| | (3.8%-43.6%) | (15.8%-89.4%) | (25.7%-68.6%) | (71.4%-100%) | |
| OS at 24 | — | — | 40.4% | 90.0% | 0.0697 |
| months | | | 95% CI | 95% CI | |
| | | | (18.3%-62.5%) | (71.4%-100%) | |

The subpopulation having high expression levels of XPG protein (2+and 3+) and the W genotype show a very clear trend (p=0.0628) towards a better outcome after treatment with ET-743 in terms of PFS and PFS6 rate (p=0.0628) and in median OS and survival at 1 and 2 years (p=0.0697). The other subpopulations have a similar outcome, clearly worse, for those patients with H and M genotype, independently of the XPG protein expression.

In fact, the subpopulation with high expression of the XPG protein and the W genotype (favourable subpopulation) has a better clinical outcome than the other 3 subpopulations (M+H genotype and low XPG protein expression, M+H genotype and high XPG protein expression and W genotype and low XPG protein expression) in terms of Median PFS (8.0 vs 3.3, 3.4 and 2.9 months, p=0.0628) and PFS>6 rate (71.1% vs. 26.3%, 27.3% and 44%, p=0.0628). The same differences are shown in terms of survival parameters, having an increased OS of 27.7 months vs 6.2, 12.2 and 11.7 months for the other three subpopulations and increased 1 year survival rates of 90% for the favourable population vs 23.7%, 52.6% and 41.7% for the other 3 subpopulations, and 2 year survival rates of 90% vs 0%, 0% and 40.4%.

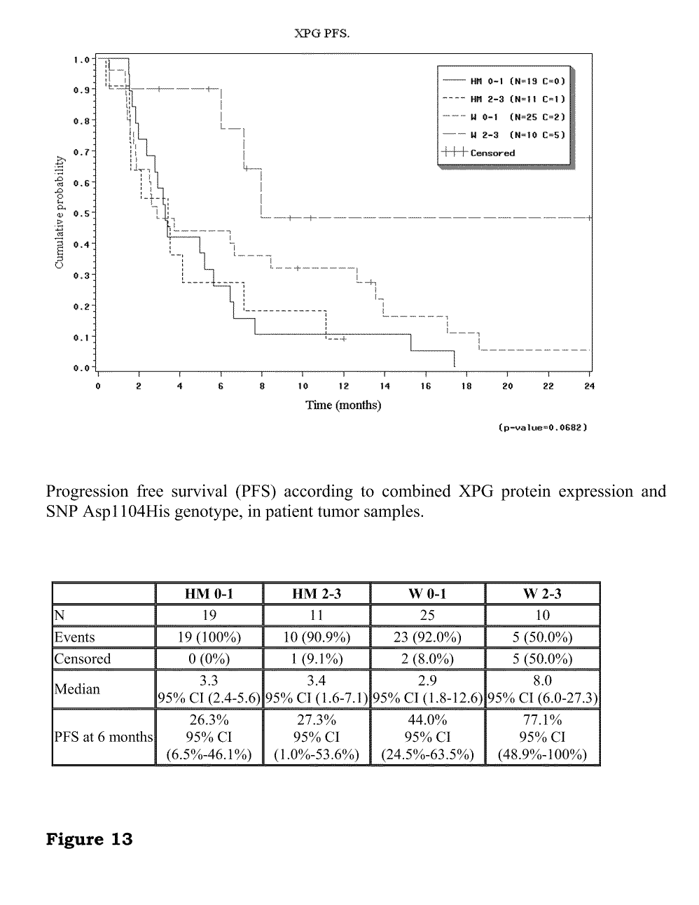

This means that, the subdivision of the full cohort of patients in four groups according to the combined expression of XPG protein and the genotype for the Asp1104His SNP distinguishes two different subpopulations according to the outcome after ET-743 treatment, ie. better and worse responders (FIGS. 12 and 13). The most favourable subpopulation having high expression of XPG protein and the W genotype with clearly a better clinical outcome compared to a less favourable subpopulation comprising the patients belonging to the other three groups defined as the combined M+H genotype and low XPG protein expression, M+H genotype and high XPG protein expression and W genotype and low XPG protein expression, respectively.

1-46. (canceled)

47. A method for predicting the clinical response of a cancer patient to ET-743 chemotherapy comprising
a) determining the expression level of XPG mRNA, or the expression level of XPG protein in a biological sample of the patient before the ET-743 chemotherapy; and
b) comparing the amount of expression of XPG mRNA in said biological sample with the median value of the expression of XPG mRNA measured in a collection of biological samples, or recording the results of the determination of the expression levels of XPG protein as negative expression (0), low expression (1+), moderate expression (2+), or high expression (3+),
wherein an expression level of XPG mRNA equal to or higher than the median value of expression levels of XPG mRNA, or a moderate or high expression level of XPG protein is indicative that the patient will show a positive response to the treatment with ET-743.

48. A method according to claim 47, further comprising the steps of:
a) determining the expression level of BRCA 1 mRNA in a biological sample of said patient before the ET-743 chemotherapy; and
b) comparing the amount of expression of BRCA1 mRNA in said biological sample with the median value of expression of BRCA1 mRNA measured in a collection of biological samples
wherein (i) an expression level of XPG mRNA equal to or higher than the median value of expression levels of XPG mRNA, or a moderate or high expression level of XPG protein and (ii) an expression level of BRCA1 mRNA lower than the median value of expression levels of BRCA1 mRNA is indicative that the patient will show a positive response to the treatment with ET-743.

49. A method according to claim 47, further comprising the step of determining the genotype of the Asp1104His SNP at locus rs17655 of the XPG gene in a biological sample of said patient before the ET-743 chemotherapy
wherein (i) an expression level of XPG mRNA equal to or higher than the median value of expression levels of XPG mRNA, or a moderate or high expression level of XPG protein and (ii) the presence of a C nucleotide in at least one of the alleles of the SNP locus rs17655 is indicative that the patient will show a positive response to the treatment with ET-743.

50. A method according to claim 48, further comprising the step of determining the genotype of the Asp1104His SNP at locus rs17655 of the XPG gene in a biological sample of said patient before the ET-743 chemotherapy
wherein (i) an expression level of XPG mRNA equal to or higher than the median value of expression levels of XPG mRNA, or a moderate or high expression level of XPG protein; (ii) an expression level of BRCA1 mRNA lower than the median value of expression levels of BRCA1 mRNA and (iii) the presence of a C nucleotide in at least one of the alleles at the SNP locus rs17655 is indicative that the patient will show a positive response to the treatment with ET-743.

51. A method of predicting the clinical response of a cancer patient to ET-743 chemotherapy comprising the step of determining the genotype of the Asp1104His SNP at locus rs17655 of the XPG gene in a biological sample of said patient before the ET-743 chemotherapy
wherein the presence of a C nucleotide in at least one of the alleles at the SNP locus rs17655 is indicative that the patient will show a positive response to the treatment with ET-743.

52. A method according to claim 51, further comprising the steps of:
a) determining the expression level of BRCA1 mRNA in a biological sample of said patient before the ET-743 chemotherapy; and
b) comparing the amount of expression of BRCA1 mRNA in said biological sample with the median value of expression of BRCA1 mRNA measured in a collection of biological samples
wherein (i) the presence of a C nucleotide in at least one of the alleles at the SNP locus rs17655 of the XPG gene and (ii) an expression level of BRCA1 mRNA lower than the median value of expression levels of BRCA1 mRNA is indicative that the patient will show a positive response after treatment with ET-743.

53. An in vitro method for designing an individual chemotherapy for a human patient suffering from cancer, comprising:
a) determining the expression level of XPG mRNA, or the expression level of XPG protein in a biological sample from said patient;
b) comparing the expression level of XPG mRNA obtained in a) with the median value of the expression level of XPG mRNA measured in a collection of biological samples, or recording the results of the determination of the expression levels of XPG protein as negative expression (0), low expression (1+), moderate expression (2+), or high expression (3+); and
c) selecting a chemotherapy treatment based on ET-743 when said XPG mRNA expression level is equal to or above the median value of expression levels of XPG mRNA, or when said XPG protein expression level has a value of (2+) or (3+).

54. A method according to claim 53, further comprising the steps of:
a) determining the expression levels of BRCA1 mRNA in a biological sample from said patient;
b) comparing the expression level of BRCA1 mRNA obtained in a) with the median value of the expression level of BRCA1 mRNA measured in a collection of biological samples; and
c) selecting a chemotherapy treatment based on ET-743 when (i) XPG mRNA expression level is equal to or above the median value of expression levels of the XPG mRNA, or when the XPG protein expression level has a value of (2+) or (3+) and (ii) the BRCA1 mRNA expression level is below the median value of expression levels of the BRCA1 mRNA.

55. A method according to claim 53, further comprising the step of determining the genotype of the Asp1104His SNP at locus rs17655 of the XPG gene in a biological sample from said patient and selecting a chemotherapy treatment based on ET-743 when (i) XPG mRNA expression level is equal to or above the median value of expression levels of XPG mRNA, or when the XPG protein expression level has a value of (2+) or (3+) and (ii) a C nucleotide is present in at least one allele at the SNP locus rs17655.

56. A method according to claim 54, further comprising the step of determining the genotype of the Asp1104His SNP at locus rs17655 of the XPG gene in a biological sample from said patient and selecting a chemotherapy treatment based on ET-743 when (i) XPG mRNA expression level is equal to or above the median value of expression levels of XPG mRNA, or when the XPG protein expression level has a value of (2+) or (3+); (ii) the BRCA1 mRNA expression level is below the median value of expression levels of the BRCA1 mRNA and (iii) a C nucleotide is present in at least one of the alleles at the SNP locus rs17655.

57. An in vitro method for designing an individual chemotherapy for a human patient suffering from cancer, comprising:
a) determining the genotype of the Asp1104His SNP at the rs17655 loci of the XPG gene in a biological sample from said patient; and
b) selecting a chemotherapy treatment based on ET-743 when a C nucleotide is present in at least one of the alleles at said SNP locus.

58. A method according to claim 57, further comprising:
a) determining the expression level of BRCA 1 mRNA in a biological sample of said patient before the ET-743 chemotherapy;
b) comparing the amount of expression of BRCA1 mRNA in said biological sample with the median value of expression of BRCA1 mRNA measured in a collection of biological samples; and
c) selecting a chemotherapy treatment based on ET-743 when (i) a C nucleotide is present in at least one of the alleles at said SNP locus and (ii) the BRCA1 mRNA expression level is below the median value of expression levels of BRCA1 mRNA.

59. A method according to claim 47 or 53, wherein the cancer from which the patient is suffering is selected from sarcoma, leiomyosarcoma, liposarcoma, osteosarcoma, ovarian cancer, breast cancer, melanoma, colorectal cancer, mesothelioma, renal cancer, endometrial cancer and lung cancer.

60. A method of treating cancer in a human patient, comprising: determining the expression levels of XPG protein or XPG mRNA in a biological sample from said patient and treating the patient with ET-743 if said expression level of XPG mRNA is equal to or higher than the median value of expression levels of XPG mRNA or if said XPG protein expression levels are higher than a reference level.

61. A method according to claim 60, further comprising the step of determining the expression level of BRCA1 mRNA in a biological sample from said patient and treating the patient with ET-743 if said expression level of XPG mRNA is equal to or higher than the median value of expression levels of XPG mRNA or if said XPG protein expression levels are higher than a reference level and if the expression level of BRCA1 mRNA is lower than the median value of expression levels of BRCA1 mRNA.

62. A method according to claim 60, further comprising the step of determining the genotype of the Asp1104His SNP at locus rs17655 of the XPG gene in a biological sample from said patient and treating the patient with ET-743 if said expression level of XPG mRNA is equal to or higher than the median value of expression levels of XPG mRNA or if said XPG protein expression levels are higher than a reference level and if the sample carries the Asp SNP genotype in at least one of the alleles of the XPG gene.

63. A method according to claim 61, further comprising the step of determining the genotype of the Asp1104His SNP at locus rs17655 of the XPG gene in a biological sample from said patient and treating the patient with ET-743 if the expression level of XPG mRNA is equal to or higher than the median value of expression levels of XPG mRNA or if said XPG protein expression levels are higher than a reference level of XPG mRNA, if the expression level of BRCA1 mRNA is lower than the median value of expression levels of BRCA1 mRNA and if he carries the Asp1 104His SNP genotype in at least one of the alleles of the XPG gene.

64. A method of treating cancer in a human patient, comprising determining the genotype of the Asp1104His SNP at locus rs17655 of the XPG gene in a biological sample from said patient and treating the patient with ET-743 if he carries the Asp1104His SNP genotype in at least one of the alleles of the XPG gene.

65. A method according to claim 64, further comprising the step of determining the expression level of BRCA1 mRNA in a biological sample from said patient and treating the patient with ET-743 if he carries the Asp1104His SNP genotype in at least one of the alleles of the XPG gene and if the expression level of BRCA 1 mRNA is lower than the median value of expression levels of BRCA1 mRNA.

66. A method according to claim 60, wherein the cancer to be treated is selected from sarcoma, leiomyosarcoma, liposarcoma, osteosarcoma, ovarian cancer, breast cancer, melanoma, colorectal cancer, mesothelioma, renal cancer, endometrial cancer and lung cancer.
